(19) United States
(12) Patent Application Publication
(10) Pub. No.: US 2023/0268081 A1
(43) Pub. Date: Aug. 24, 2023

(54) METHODS AND SYSTEMS FOR AUTOMATICALLY POPULATING A USER INTERFACE OF AN ELECTRONIC HEALTH RECORDS SYSTEM WITH CLINICALLY-RELEVANT ACTIONABLE PATIENT DATA

(71) Applicant: MedicineBox, Albuquerque, NM (US)

(72) Inventors: Abinash Achrekar, Albuquerque, NM (US); Shalini Shanker, Albuquerque, NM (US)

(21) Appl. No.: 18/107,921

(22) Filed: Feb. 9, 2023

Related U.S. Application Data
(60) Provisional application No. 63/311,971, filed on Feb. 19, 2022.

(51) Int. Cl. G16H 70/20; G16H 10/60

(52) U.S. Cl. CPC G16H 70/20; G16H 10/60

(57) ABSTRACT

A method and system for self-populating a user interface with medical data, can involve transforming patient data associated with a patient into clinically-relevant actionable patient data that is relevant to the patient based on a diagnosis of the patient and which is displayable in a screen of a user interface of an electronics health records (EHR) system in a critical diagnosis hierarchy relevant to the diagnosis of the patient. In response to a user input, a user interface screen can be automatically populated with the clinically-relevant actionable patient data according to the critical diagnosis hierarchy and the diagnosis in a manner that minimizes user input with respect to the user interface.

### CROSS-REFERENCE TO PROVISIONAL APPLICATION

This patent application claims priority under 35 U.S.C. § 119(e) to U.S. Provisional Pat. Application Serial No. 63/311,971 entitled “Methods and Systems for Automatically Populating a User Interface of an Electronic Health Records System with Clinically-Relevant Actionable Patient Data,” which was filed on Feb. 19, 2022, the disclosure of which is incorporated herein by reference in its entirety.

### TECHNICAL FIELD

Embodiments are related to improvements in data-processing systems and methods. Embodiments further relate to the field of electronic medical records data management, deployment, diagnosis, and display through a data-processing system. Embodiments also relate to electronic health record (EHR) systems and user interfaces that enable EHR data retrieval, organization and display.

### BACKGROUND

There are currently over 700 federally certified electronic health record (““EHR”) vendors. Electronic medical records compiled in the form of EHRs were intended to bring higher-quality care and save costs by harnessing ‘big data’. This did not happen, however, in practice. Every provider (e.g., hospital or clinic) chooses an EHR for their respective practice, which may be isolated from other EHRs. Patients often move between medical providers, and when they do practitioners cannot timely access patient data remotely or without patient intervention. The practitioner must then request patient health records from previous health providers. This can take an average of three days or more and the patient’s information may arrive on hundreds of pages by fax or CD-ROM. Furthermore, these pages may not be well organized and may essentially be considered what amount to mostly a “data dump” of information that must be sorted through by its recipients.

Medical staff may spend hours on the phone, for example, to obtain information from other clinics arriving via fax or by mail. Waiting three days to take action during an emergency is simply not an option. The result of such a delay can often result in repeat testing, increased admissions, increased hospitalization, increased costs, and, ultimately, a loss of profit and poor patient outcomes.

Additionally, current file sharing systems are cumbersome and inundated with too much superfluous information. For example, with conventional EHR systems, doctors may spend half their day ‘clicking’ through EHR’s looking for specific and actionable data instead of interacting with patients. In January 2019, the Harvard School of Public Health and Massachusetts Medical Society called the use of EHR a “public health crisis”.

To solve these problems, what is needed is a new approach to providing doctors and other healthcare providers and professionals with instant access to medical records. What is also needed is a new approach to provide doctors and healthcare providers and professionals with access to medical records in an organized presentation, and which can also enable (e.g., medical data from EHRs) to be organized on demand based on a recipient’s selection of condition-related data. What is also needed is a system that can automatically rank prescriptions based on their relevance to conditions that can be selected by a reviewer for display on a user interface. Conventional EHR systems do not adequately address these problems, resulting in an ongoing loss of time and increased costs for both the patients and their healthcare providers, and the potential for patient harm.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is, therefore, one aspect of the embodiments to provide an improved user interface for the display of patient medical data extracted from electronic health records (EHRs).

It is another aspect of the embodiments to provide for a method and system for extracting clinically-relevant actionable patient data from multiple EHRs for display through a simple graphical user interface.

It is further an aspect of the embodiments to provide for methods and systems for self-populating a user interface with medical data extracted from EHRs.

It is another aspect of the embodiments to provide a user interface for an EHR data retrieval system that can display critical diagnosis hierarchy relevant to the diagnosis of the patient.

It is another aspect of the embodiments to provide a user interface for an EHR system that can display prescription drugs in a hierarchy relevant to a condition selected for review by a record reviewer.

It is yet another aspect of the embodiments to provide a user interface for the organization and display of data extracted from records stored in EHR forms and systems that minimizes the user input necessary to retrieve and display clinically-relevant actionable patient data.

It is another aspect of the embodiments to provide a user interface for the organization and display of data extracted from records stored in EHR systems that reacts to user input in a manner to enable the retrieval and display of clinically-relevant actionable patient data that is based on a particular medical condition selected from a plurality of medical conditions listed in the user interface in association with a patient.

It is another aspect of the embodiments to provide a user interface for the organization and display of data extracted from records stored in EHR systems, which can react to a user input to retrieve and display hierarchically ordered pharmaceutical prescription information that is based on a particular medical condition that can be selected from a plurality of medical conditions listed in the user interface in association with a patient.

The aforementioned aspects and other objectives and advantages can now be achieved as described herein. In an embodiment, a method for self-populating a user interface with medical data, can involve transforming patient data associated with a patient into clinically-relevant actionable patient data that is relevant to the patient based on at least one diagnosis of the patient and which is displayable in a screen of a user interface of an electronics health records (EHR) system in a critical diagnosis hierarchy relevant to the at least one diagnosis of the patient, and in response to a user input, automatically populating the screen of the user interface with the clinically-relevant actionable patient data according to the critical diagnosis hierarchy and the at least one diagnosis in a manner that minimizes the user input with respect to the user interface.

An embodiment can involve fetching the patient data associated with the patient and based on the at least one diagnosis of the patient.

An embodiment can involve fetching the patient data associated with the patient and based on the at least one diagnosis of the patient prior to transforming the patient data into the clinically-relevant actionable patient data.

In an embodiment, the at least one diagnosis can be derived from a medical ontology.

In an embodiment, the user interface can be implemented in the context of a touch-sensitive display.

In an embodiment, the clinically-relevant actionable patient data displayed in the user interface can include one or more of, for example, imaging data relevant to the patient, medical images relevant to the patient, test results relevant to the patient, laboratory results relevant to the patient, medical allergies relevant to the patient, and prescriptions relevant to the patient.

In an embodiment, a method for self-populating a user interface with medical data, can involve: deriving at least one diagnosis of a patient from a medical ontology; transforming patient data associated with the patient into clinically-relevant actionable patient data that is relevant to the patient based on the at least one diagnosis of the patient and which is displayable in a screen of a user interface of an electronics health records (EHR) system in a critical diagnosis hierarchy relevant to the at least one diagnosis of the patient; and automatically populating the screen of the user interface with the clinically-relevant actionable patient data according to the critical diagnosis hierarchy and the at least one diagnosis in a manner that minimizes user input with respect to the user interface.

In an embodiment, a method for self-populating a user interface with medical data, can include at least one processor and a memory storing processor-executable codes, wherein the at least one processor is configured to implement the following operations upon executing the processor-executable codes: transforming patient data associated with a patient into clinically-relevant actionable patient data that is relevant to the patient based on at least one diagnosis of the patient and which is displayable in a screen of a user interface of an electronics health records (EHR) system in a critical diagnosis hierarchy relevant to the at least one diagnosis of the patient; and in response to a user input, automatically populating the screen of the user interface with the clinically-relevant actionable patient data according to the critical diagnosis hierarchy and the at least one diagnosis in a manner that minimizes the user input with respect to the user interface.

In an embodiment, the processor-executable codes can be configured to fetch the patient data associated with the patient and based on the at least one diagnosis of the patient.

In an embodiment, the processor-executable codes can be configured to fetch the patient data associated with the patient and based on the at least one diagnosis of the patient prior to transforming the patient data into the clinically-relevant actionable patient data.

In an embodiment, a system for self-populating a user interface with medical data, can include at least one processor and a memory storing processor-executable codes, wherein the at least one processor is configured to implement the following operations upon executing the processor-executable codes: deriving at least one diagnosis of a patient from a medical ontology; transforming patient data associated with the patient into clinically-relevant actionable patient data that is relevant to the patient based on the at least one diagnosis of the patient and which is displayable in a screen of a user interface of an electronics health records (EHR) system in a critical diagnosis hierarchy relevant to the at least one diagnosis of the patient; and automatically populating the screen of the user interface with the clinically-relevant actionable patient data according to the critical diagnosis hierarchy and the at least one diagnosis in a manner that minimizes user input with respect to the user interface.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a flow chart depicting logical operational steps of a method for providing a user interface for an electronic health records (EHRs) data retrieval system, in accordance with an embodiment;

FIG. 2 illustrates a flow chart depicting logical operational steps of a method for providing a user interface for an electronic health records (EHRs) data retrieval system, in accordance with another embodiment;

FIG. 3 illustrates a flow chart depicting logical operational steps of a method for populating a user interface with clinically-relevant actionable data obtained from electronic health records (EHRs), in accordance with another embodiment;

FIG. 4 illustrates a block diagram of a cloud-based system for extracting and displaying clinically relevant actionable patient data from multiple electronic medical records, in accordance with an embodiment;

FIG. 5 illustrates a block diagram of an example computing device that can implement the features, methods, systems, and processes of the embodiments;

FIG. 6 illustrates a screenshot of a user interface in accordance with an embodiment;

FIG. 7 illustrates a screen shot of a user interface in accordance with an embodiment;

FIG. 8 illustrates a flow diagram depicting user flow for a user interface in which clinically-relevant actionable patient data is automatically populated in response to minimal user input, in accordance with an embodiment;

FIG. 9 illustrates a flow chart of operations depicting logical operational steps of a method for providing a user interface for an electronic health records (EHR) data retrieval system, in accordance with an embodiment;

FIG. 10 illustrates a flow chart of operations depicting logical operational steps of a method for implementing a rule engine, in accordance with an embodiment;

FIG. 11 illustrates a flow chart of operations depicting logical operational steps of a method for implementing a rule engine, in accordance with an embodiment;

FIG. 12 illustrates a flow chart of operations depicting logical operational steps of a method for implementing a rule engine, in accordance with an embodiment;

FIG. 13 illustrates a flow chart of operations depicting logical operational steps of a method for implementing a transformation process, in accordance with an embodiment;

FIG. 14 illustrates a flow chart of operations depicting logical operational steps of a method for implementing a transformation process, in accordance with an embodiment;

FIG. 15 illustrates a diagram depicting a user interface and associated user interface features, in accordance with an embodiment;

FIG. 16 illustrates various types of data that can be displayed by a user interface, in accordance with an embodiment; and

FIG. 17 illustrates additional types of data that can be displayed by a user interface, in accordance with an embodiment.

Like reference numerals or reference symbols in the various drawings may indicate like or similar elements.

### DETAILED DESCRIPTION

The particular values and configurations discussed in these non-limiting examples can be varied and are cited merely to illustrate one or more embodiments and are not intended to limit the scope thereof.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” or “in an embodiment” and variations thereof as utilized herein do not necessarily refer to the same embodiment and the phrase “in another embodiment” or “in another example embodiment” and variations thereof as utilized herein may or may not necessarily refer to a different embodiment. It is intended, for example, that claimed subject matter can include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Several aspects of data-processing systems will now be presented with reference to various systems and methods. These systems and methods will be described in the following detailed description and illustrated in the accompanying drawings by various blocks, modules, components, circuits, steps, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. Note that the term ‘data-processing system’ or ‘data-processing apparatus’ as utilized herein can relate to a combination of machines, people, and processes that for a set of inputs produces a defined set of outputs. The inputs and outputs can be interpreted as data, facts, information etc. depending on the interpreter’s relation to the system. Examples of a data-processing system or a data-processing apparatus can include a computing device, a server, a mobile computing device, or a combination of computing devices and components and modules (which may be hardware modules, software modules and/or combinations thereof).

By way of example, an element, or any portion of an element, or any combination of elements may be implemented with a “processing system”, a “data processing system” or a “data-processing apparatus” that can include one or more processors. Examples of processors can include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. One or more processors in the processing system or apparatus may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. A mobile “app” is an example of such software.

The term ‘processor’ as utilized herein can relate to an integrated electronic circuit that performs calculations that run a computer. A processor can perform arithmetical, logical, input/output (I/O) and other basic instructions that can be passed from an operating system (OS). Most other processes are dependent on the operations of a processor. The terms processor, CPU and microprocessor are commonly linked and may be utilized interchangeably with one another to refer to the same device, system or circuit.

Accordingly, in one or more exemplary embodiments, the functions described may be implemented in hardware, software, firmware, or any combination thereof. If implemented in software, the functions may be stored on or encoded as one or more instructions or code on a computer-readable medium. Computer-readable media includes computer storage media. Storage media may be any available media that can be accessed by a computer.

By way of example, and not limitation, such computer-readable media can include read-only memory (ROM) or random-access memory (RAM), electrically erasable programmable ROM (EEPROM), including ROM implemented using a compact disc (CD) or other optical disk storage, magnetic disk storage or other magnetic storage devices, or any other medium that can be used to carry or store desired program code in the form of instructions or data structures and that can be accessed by a computer. The term ‘disk’ and ‘disc’, as used herein, can include CD, laser disc, optical disc, digital versatile disc (DVD), and floppy disk where disks usually reproduce data magnetically, while discs reproduce data optically with lasers. Combinations of the above should also be included within the scope of computer-readable media. The term ‘computer-readable media’ can include storage devices such as flash drives or flash memory, and other types of memory devices.

The following discussion is intended to provide a brief, general description of suitable computing environments in which the system and method may be implemented. Although not required, the disclosed embodiments can be described in the general context of computer-executable instructions, such as program modules, being executed by a single computer. In most instances, a “module” (also referred to as an “engine”) may constitute a software application but can also be implemented as both software and hardware (i.e., a combination of software and hardware).

Generally, program modules include, but are not limited to, routines, subroutines, software applications, programs, objects, components, data structures, etc., that perform particular tasks or implement particular data types and instructions. Moreover, those skilled in the art will appreciate that the disclosed method and system may be practiced with other computer system configurations, such as, for example, hand-held devices, multi-processor systems, data networks, microprocessor-based or programmable consumer electronics, networked PCs, minicomputers, mainframe computers, servers, and the like.

Note that the term module (or an engine) as utilized herein may refer to a collection of routines and data structures that perform a particular task or implements a particular data type. Modules may be composed of two parts: an interface, which lists the constants, data types, variable, and routines that can be accessed by other modules or routines, and an implementation, which is typically private (accessible only to that module), and which includes source code that actually implements the routines in the module. The term module may also simply refer to an application, such as a computer program designed to assist in the performance of a specific task, such as word processing, accounting, inventory management, etc.

In some embodiments, the term “module” can refer to a modular hardware component or a component or element that can be a combination of hardware and software. It should be appreciated that implementation and processing of such modules according to the approach described herein can lead to improvements in processing speed and ultimately in energy savings and efficiencies in a data-processing system or apparatus. A “module” can thus perform the various steps, operations or instructions discussed herein to achieve the aforementioned improvements in processing speed and energy savings and efficiencies in a data-processing system.

The embodiments provide for a health analytics tool that can scour and scrape multiple electronic health records (EHRs) for clinically relevant patient data. This approach can allow medical personnel and patients quick and easy access to vital, prioritized and actionable medical records from any hospital across the United States. The term “MedicineBox” or “Medicine Box” as utilized herein can relate to an improved electronic graphical user interface (GUI) dashboard for displaying data extracted from EHRs of a patient, and as discussed herein, facilitate the move away from ‘big data’ to small specific relevant actionable data.

A team of doctors can create a list of top problems that have the most frequent consults or occur in an emergency, for example, and compile the limited information needed to take immediate action. With one ‘click’ of a mouse or touch screen display, the disclosed “MedicineBox” module can extract only this information from numerous electronic health record platforms and display the top problems and top indicators for each patient on one screen. This approach thus stores only the latest result and automatically overwrites itself solving the issue of massive medical record storage.

Note that the terms ‘click’ and variations thereof (e.g., ‘clicking’) as utilized herein can relate to a user interface (e.g., computer interface such as a GUI) that allows the activation of a file or function by selection with a pointing device (e.g., such as a mouse, touch pad, touch screen, etc.). The term ‘click’ and ‘clicking’ can relate to a type of user input that a user may invoke or use to input data through the user interface. For example, a click may refer to the carrying out of a computer operation by pressing (‘clicking’) a button on a mouse, keypad, keyboard or other input device (e.g., touch screen display). The terms ‘click’ and ‘clicking’ and variations thereof may also relate to ‘point-and-click,” which allows for the activation of commands moving a graphically displayed cursor over certain user interface areas or icons and ‘clicking’ with a pointing device. Thus, ‘click’ may relate to a user input such as a button click or a mouse click.

A ‘1-click’ or ‘one-click’ user input, for example, may be utilized in some embodiments as a user input, which can result in, for example, automatically populating the screen of a user interface with clinically-relevant actionable patient data according to a critical diagnosis hierarchy and a medical diagnosis of a patient in a manner that minimizes the user input with respect to the user interface, as discussed hereins.

The embodiments thus relate to methods and systems for self-populating a user interface with medical data and can involve operations for transforming patient data associated with a patient into clinically-relevant actionable patient data that is relevant to the patient based on a diagnosis of the patient and which is displayable in a screen of a user interface of an electronics health records (EHR) system in a critical diagnosis hierarchy relevant to the diagnosis of the patient. In response to a user input, a screen of the user interface can be automatically populated with the clinically-relevant actionable patient data according to the critical diagnosis hierarchy and the diagnosis in a manner that minimizes user input with respect to the user interface.

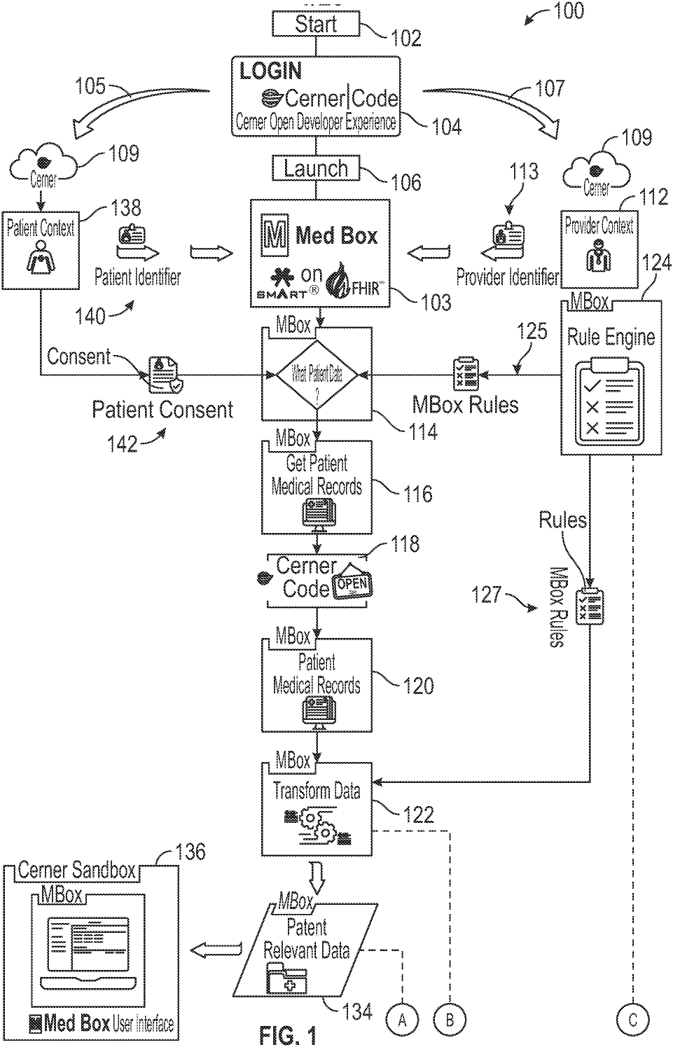

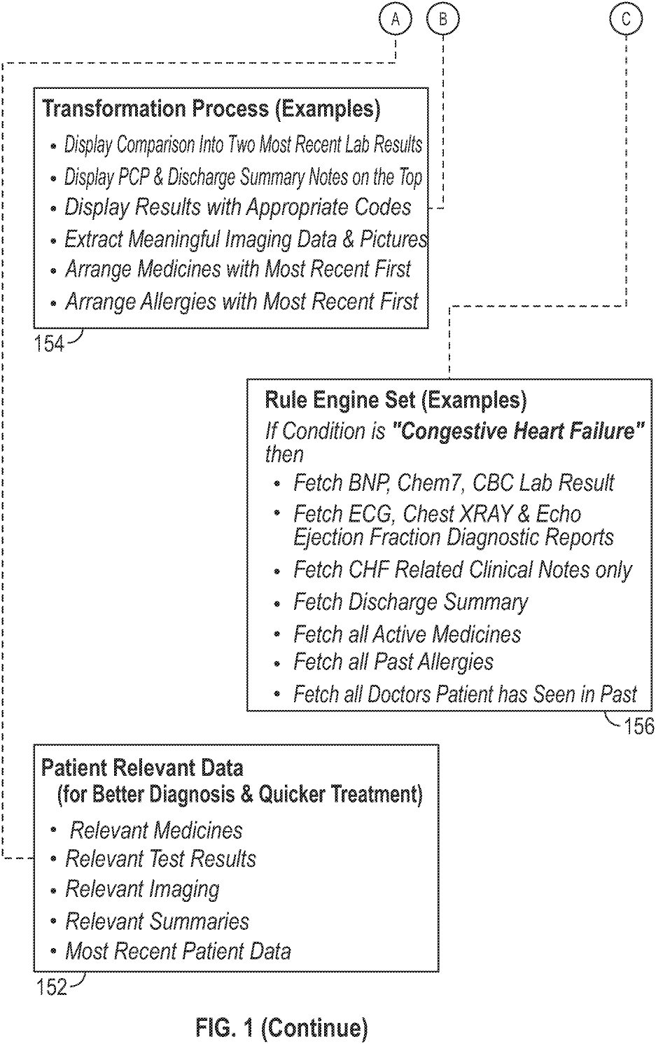

FIG. 1 illustrates a flow chart of operations depicting logical operational steps of a method 100 for providing a user interface for an electronic health records (EHR) data retrieval system, in accordance with an embodiment. As shown at block 102, the process can begin. Next, as depicted at block 104, a login operation can be implemented using a computer platform such as (but not limited to) the Cerner Open Developer Experience (Code), also referred to in some cases as “code_”. Code is a software product created by the Cerner Corporation, which can enhance collaboration with third-party and client developers on SMART® on FHIR® applications. code_ is a digital experience designed to meet market expectations of open communications, robust API documentation, provide clearly defined guidelines and access to tools that enable innovative app development.

Any developer may visit the website, code.cerner.com, to begin coding immediately with the SMART® on FHIR® tools available in the Cerner open sandbox platform, and/or with research educational development tools, and also to browse current ‘apps’ that are available or in development through this website. The website is mobile-friendly, so users can access it from a number of different devices. Thus, a user can log into the code_ software and then launch the MB (Medicine Box) module 103 (labeled as ‘MedBox’ in FIG. 1).

As will be discussed in greater detail herein the module 103 can be used as part of an EHR system to display patient medical data extracted from EHRs. The module 103 can be provided with patient data including a patient identifier as shown at arrow 140 which can be derived from patient context data, as depicted at block 138. The module 103 can also be provided with a provider identifier as indicated at arrow 113, which is obtained from provider context data as shown at block 112. Both the patient context data and the provider context data can be obtained from a cloud network 109, which in this example can be the Cerner cloud network.

As shown next at decision block 114, a step or operation can be implemented to determine what patient data should be obtained. This decision is based on rules 125 obtained from a rule engine 124. This decision is also based on patient consent 142, which in turn is obtained from the patient context data 138. Following processing of the operation depicted at block 116, a step or operation can be implemented to obtain the patient medical records. Cerner code_ can be used to open the patient medical records, as depicted at block 118.

Thereafter, the patient medical records are obtained, as shown at block 120 and the subject to a data transformation operation as illustrated at block 122. Note that rules 127 obtained from the rule engine 124 can be used to implement the data transformation operation shown at block 122. Examples of the data transformation process for the data transformation operation are shown at block 154. For example, the data transformation operation/process may involve configuring the patient medical data for a display as a comparison between the patient’s two most recent lab results, a display of a PCP with discharge notes in a particular section (e.g., top portion) of a GUI screen, a display of results with appropriate medical codes, an extraction of meaningful imaging data and/or other pictures associated with the patient, an arrangement of prescription medicines with the most recent first, an arrangement of patient allergies with the most recent first, and so on. Although not depicted at block 154, medicines can also be arranged hierarchically based on a condition chosen in association with a patient. Note that the GUI or graphical user interface as utilized herein can relate to a user interface that can allow users to interact with different electronic devices using icons and other visual indicators and objects graphically displayed in the GUI.

Similarly, examples of a rule engine set for the rule engine 124 are shown at block 156. The example shown at block 156 relates to “Congestive Heart Failure”. If “Congestive Heart Failure” is a condition or a diagnosis, possible rules for this particular condition or diagnosis may be fetching a BNP, Chem 7, CBS lab result, fetching ECG, chest x-ray(s) and echo ejection fraction diagnostic reports, fetching CHF related clinical notes, fetching a patient discharge summary, fetching all active medicines associated with the patient, fetching all past allergies associated with the patient, fetching the names and data of all doctors that patient has seen in the past, and so on. Although not indicated at block 156, medicines can also be arranged hierarchically based on a condition selected in association with a patient. So, in the given example of congestive heart failure, medicines can be arranged in an order of importance or relevance for this condition.

Following completion of the data transformation operation shown at block 122, clinically-relevant actionable data, can be generated as depicted at block 134 and then self-populated in a screen of a user interface as shown at block 136. The data transformation operation includes configuring or formatting the data into data that is relevant to the diagnosis of the patient and also which can be displayed in a screen of a user interface associated with an EHR system in a critical diagnosis hierarchy relevant to the diagnosis of the patient. The user interface can be a display screen in a client used by a practitioner at a medical facility. A client can include any computer system typical accessed by medical providers at facilities, including desktop computer, portable computers, wireless handheld devices with secured connections to facility data networks.

Note that some examples of clinically-relevant actionable data are shown in block 152. For example, clinically-relevant actionable data may include a list of medicines that are relevant to the patient and to the patient’s diagnosis, relevant test results, relevant patient summary information, and most recent patient data.

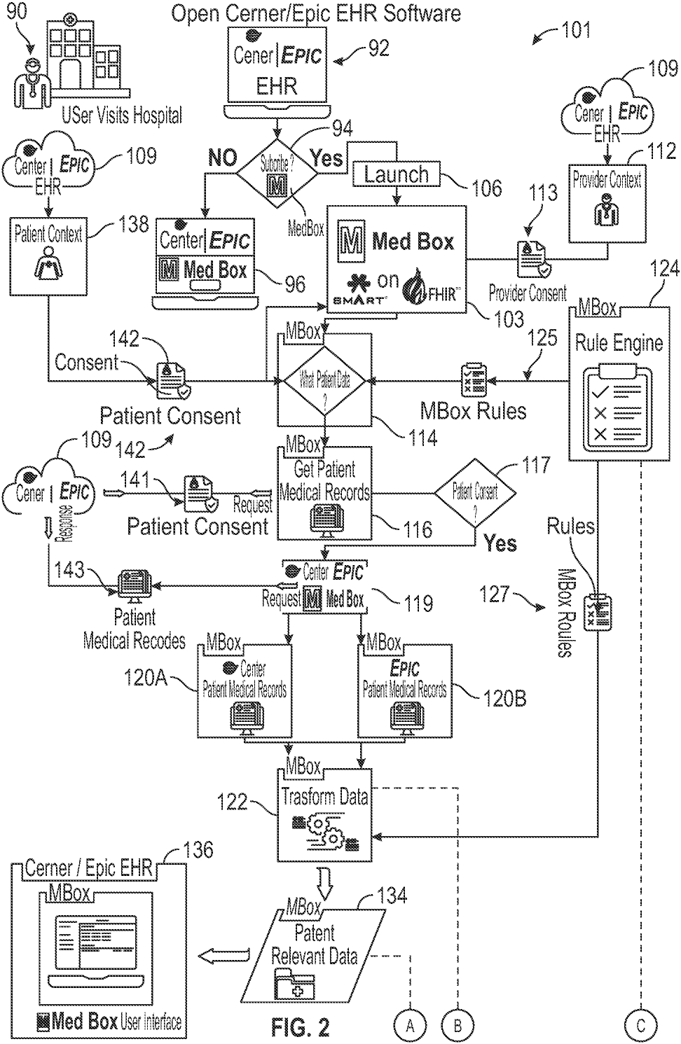

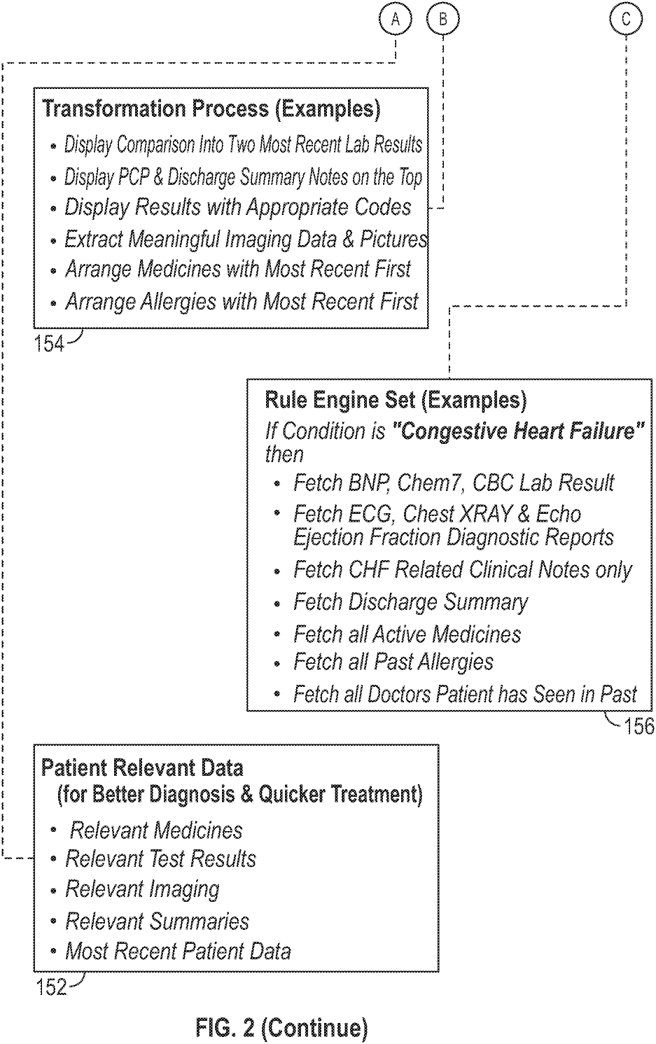

FIG. 2 illustrates a flow chart of operations depicting logical operational steps of a method for providing a user interface for an EHR data retrieval system, in accordance with another embodiment. Note that the method 101 shown in FIG. 2 is similar to the method 100 depicted in FIG. 1 albeit with some minor differences. For example, as shown at block 90 in FIG. 2, a patient may visit a hospital and as shown thereafter, a user (e.g., a physician, nurse practitioner, etc.) may access a medical software platform and/or software application through, for example, a software platform or software application such as open Cerner and/or Epic EHR software through a client device 92 to input and view medical data associated with the patient.

Note that the term “Epic” refers to an electronic medical record (EMR) system, practice management software, and/or hospital information systems for mid-sized and large medical groups, hospitals, and integrated healthcare organizations. It should be appreciated that any reference to specific software applications and platforms, such as “Epic” software/platforms or “Cerner” software such as code_ herein is for illustrative and exemplary purposes only and should not be considered limiting features of the embodiments.

A user may subscribe to the module 103 (“MedicineBox”) as shown at decision block 94, resulting in either a launch (YES) of the module 103 as shown at block 106 or simply a screen inviting the user to subscribe (NO) as shown at block 96. Another difference between the method 101 shown in FIG. 2 and the method 100 depicted in FIG. 1 involves a patient consent 141 in response to a request to obtain the patient medical records (see block 116). Patient consent must be provided for access to the cloud network 109 and access to patient medical records, as shown at block 143. An additional patient consent operation can also be implemented following the request to get medical records, as shown at decision block 117. Assuming a “YES” answer, the medicine box module 103 can be accessed, as shown at block 119 with a request for the patient medical records (see block 143) with respect to the cloud network 109. Patient medical records can be accessed as shown at block 120A and 120B and then subject to the data transformation operation depicted at block 122.

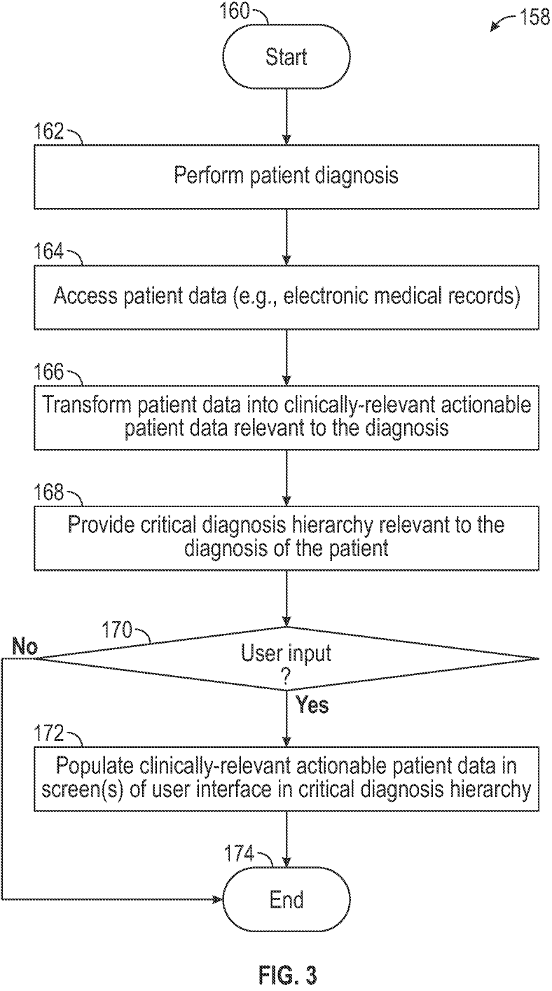

FIG. 3 illustrates a flow chart of operations depicting logical operational steps of a method 158 for populating a user interface with clinically-relevant actionable data, in accordance with another embodiment. As shown at block 160, the process can begin. Thereafter, as indicated at block 162, a step or operation can be implemented in which a diagnosis (medical diagnosis) of a patient can be performed.

Next, as shown at block 164, patient data (electronic medical records associated with the patent) can be accessed. Thereafter, as shown at block 166, a step or operation can be implemented to transform the patient data into clinically-relevant actionable patient data that is relevant to the diagnosis, and which can be displayable in a screen of a user interface of an electronics health records (EHR) system in a critical diagnosis hierarchy relevant to the diagnosis of the patient. Block 168 illustrates a step or operation in which the actual critical diagnosis hierarchy relevant to the diagnosis of the patient is provided or generated. Any display of data (including population of data) in a user interface screen will be displayed/populated based on the critical diagnosis hierarchy relevant to the diagnosis of the patient.

Then, as shown at decision block 170, a step or operation can be implemented to determine if a user input has occurred. If a user input has occurred, then, as indicated at block 172, a step or operation can be implemented in which the transformed patient data (i.e., clinically-relevant actionable patient data) is automatically populated in a screen (or screens) of the user interface with the clinically-relevant actionable patient data according to the critical diagnosis hierarchy and the at least one diagnosis in a manner that minimizes the user input with respect to the user interface.

Note that the diagnosis (i.e., the results of the patience diagnosis) may be based at least in part on a medical ontology and/or can be derived from the medical ontology. Note that the term ‘ontology’ as utilized herein can relate to a dynamic, controlled vocabulary for describing a plurality of objects or concepts contained in a process or domain, and their interrelationships. An ontology may be further thought of as a formal knowledge-representation system which comprises rules for encoding the manner in which knowledge is represented, along with rules that enable automated reasoning with regard to the objects or concepts represented.

An ontology term may be a single named concept describing an object or entity. A concept may, for example, comprise a collection of words and associated relevance weights, co-occurrence and word localization statistics that describe a topic.

The data transformation step or operation depicted at block 166 in FIG. 3 and also in block 122 in FIG. 1 and FIG. 2 can involve the extraction of ‘meaningful’ imaging data, pictures and other information from the patient’s medical records. Data transformation can include the use of natural language processing (NLP) including NLP rules in the rule engine 124 (see FIG. 1 and FIG. 2). NLP can thus be employed as part of the data transformation processing including the extraction operation discussed above. NLP relates to the branch of computer science, and more specifically, the branch of artificial intelligence (Al) concerned with giving computers the ability to understand text and spoken words in much the same way human beings can.

NLP combines computational linguistics-rule-based modeling of human language-with statistical, machine learning, and deep learning models. Together, these technologies can enable computers to process human language in the form of text or voice data and to ‘understand’ its full meaning, complete with the speaker or writer’s intent and sentiment. NLP can include speech recognition, part of speech tagging, word sense disambiguation, named entity recognition, co-reference resolution, sentiment analysis, and/or natural language generation.

A non-limiting example of NLP that can be adapted for use with an embodiment to extract meaningful imaging data, pictures and other information from the patient’s medical records as part of the data transformation process is disclosed in U.S. Pat. Application No. 20210342380, entitled “Generative Ontology Learning and Natural Language Processing with Predictive Language Models,” which published on Nov. 4, 2021 and is incorporated herein by reference in its entirety.

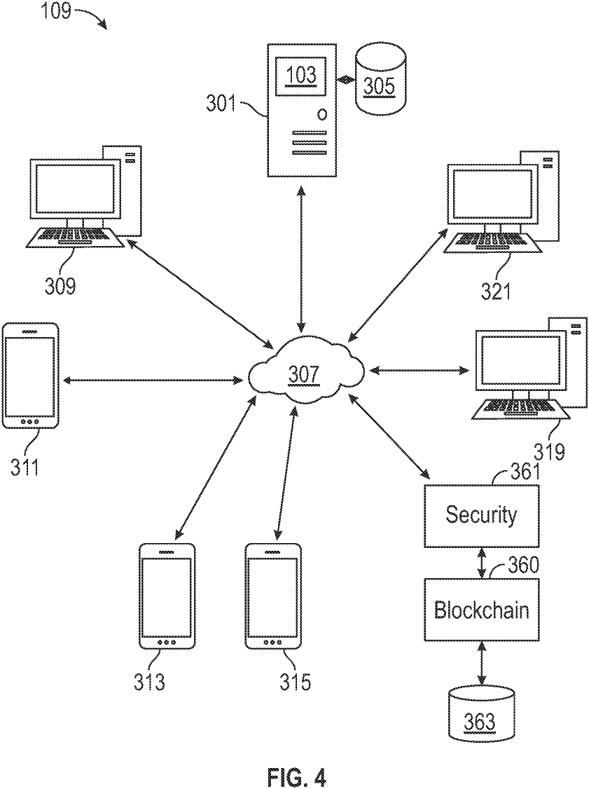

FIG. 4 illustrates a block diagram of a cloud-based system 109 for extracting and displaying clinically relevant actionable patient data from multiple electronic medical records, in accordance with an embodiment. The cloud-based system 109 can be implemented in the context of a cloud-based, blockchain, and/or HIPAA compliant module(s) that uses machine learning and Al to extract the clinically-relevant actionable patient data from multiple electronic medical records and display the transformed data with a simple user interface. Such an approach can store the latest medical test and can automatically overwrite itself, thereby solving the issue of massive medical record storage.

The term clinically-relevant actionable patient data as utilized herein can refer to data that relates to the “bedside” of a patient, the course of his or her disease and/or the observation and treatment of patients directly. Such clinically-relevant actionable patient data can include patient data that a physician, a nurse, a physician’s assistant, a nurse practitioner and/or other medical personnel can utilize to chart and implement actionable treatment for the patient. The term clinically-relevant actionable patient data can also refer to data that is of clinical significance including the practical importance of a treatment effect -- whether it has a real genuine, palpable, and/or noticeable effect on daily life. Clinically-relevant actionable patient data is data that is relevant to the patient based on at least one diagnosis of the patient and which is displayable in a screen of a user interface of an EHR system in a critical diagnosis hierarchy relevant to the diagnosis of the patient.

Note that the term cloud-based as utilized herein relates to applications, services or resources made available to users on demand via the Internet from a cloud computing provider’s servers. Cloud-based computing can be utilized as a way to increase capacity, enhance functionality or add additional services on demand without having to commit to potentially expensive infrastructure costs or increase / train existing in-house support staff. In addition, HIPAA is United States legislation that provides data privacy and security provisions for safeguarding medical information.

The cloud-based system 109 can include a server 301 that can host the MB (Medicine Box) module 103 and which can provide electronic access to a database 305. The database 305 can store patient medical records, including, for example, baseline records, incident records, prescriptions, treatment programs, and so on. The database 305 can further store a plurality of access permissions, which can define which types or classes of users may read or write data to the database 305. The database 305 may be distributed, so that the access permissions and the patient medical records may be stored in different databases. Alternatively, all storage for the system may be in a single database.

In a practical application, the database 305 may be configured from different databases and repositories. For example, the database 305 may include multiple databases implemented across a distributed network. The server 301 can be linked by a network 307, such as the Internet, to one or more client devices such as computing devices 309, 321, 319 and mobile computing devices 311, 313, 315. Note that the term mobile computing device as utilized herein can refer to, for example, a smartphone, a tablet computing device, a laptop computer, a wearable computing device (e.g., Apple Watch, Google Glass, and so on), etc.

The MB module 103 can be configured to enable the various computing devices 309, 321, 319 and 311, 313, 315 depicted in FIG. 1 to access the database 305, whether to contribute or retrieve patient data, depending on the permissions related to the access profile of the user utilizing the computing device. Although the MB module 103 may run as a single utility, in embodiments, it can present as different sub-utilities, each having distinct functionalities depending on the identity and access level of the user attempting to access the MB module 103. The MB module 103 may be hosted on the server 301, as shown, and accessed by the various users as, for example, a webpage, and/or the utility can be hosted on each or some of the computing devices 309, 321, 319 and 311, 313, 315.

The computing devices 309, 321, 319 and 311, 313, 315 are shown as being either a desktop computer, a smartphone (or tablet), or a standalone server, but it will be appreciated that the devices 309, 321, 319 and 311, 313, 315 may be embodied by any suitable computing device or combination of computing devices, such as, for example, desktop computer, laptop computer, smartphone, tablet or server computer. Each computing device 309, 321, 319 and 311, 313, 315 comprises a processor and memory, the memory having stored thereon computer instructions, which when executed, can cause the device to execute tasks described herein. The MB module 103 may be accessed by a web portal generating web pages accessible by web browsers or web-enabled applications executed on devices such as computing devices 309, 321, 319 and 311, 313, 315. Note that the term portal as utilized herein can relate to a communication portal including a web-based portal accessible by computing devices such as the computing devices 309, 321, 319 and 311, 313, 315.

The network 307 may provide for wired or wireless communication between the server and the devices. Wireless communication may be provided by any suitable protocol, such as, for example, IEEE 802.11, GPRS, 3G, 4G, and LTE. The network 307 may be the Internet.

As discussed previously herein, there are currently over 700 federally certified EHR vendors. Electronic medical records were intended to bring higher-quality care and save money by harnessing big data. However, this did not happen in practice. Every hospital or clinic (providers) selects an EHR for their respective practice, which is isolated from other EHRs. When patients move between providers, practitioners cannot access patient data remotely. They must request patient health record from the provider. This can take an average of three days and the patient’s information typically arrives on hundreds of pages by fax or CD-Rom.

In addition, clinicians cannot wait three days to take action in emergency settings, which may result in repeat testing. Additionally, the current file sharing systems are too cumbersome and inundated with too much superfluous information.

The MB module 103 thus moves away from ‘big data’ to small specific relevant data. A team of doctors can create a list of key demographics and top problems that may occur in an emergency and compile and present the information they may need to take immediate action. The MB module 103 thus extracts only this information from numerous EHRs and displays the top problems and top indicators for each patient in one screen. The MB module 103 can store the latest test and can automatically overwrite itself solving the issue of massive medical record storage.

For example, if a patient has hypertension, the MB module 103 can extract the last Chem 7 labs, Urine Analysis, Electrocardiogram (ECG) and Cardiac Stress test data. This is information a doctor may need to know to assess and address the patient in a timely manner.

The MB module 103 can offer a number of improvements and advantages. For example, critical health information can be made more readily available by providing patients and doctors with access to key medical information on any patient, anytime and anywhere. Patients and/or approved family members can also share vital health information with providers via a mobile “app”.

The MB module 103 may also help to reduce so-called “doctor burnout” through the implementation of a better user experience and interface. The MB module 103 can synthesize information from numerous EHRs and display key and actionable information on a simple GUI instead of digging through massive patient databases. The information displayed helps practitioners direct care and make more specific medical searches, if necessary, from EHRs. In addition, the MB module 103 can facilitate increasing efficiency and reducing excessive repeat test by having the last or latest test data readily available. This also can reduce “upcoding” for Medicaid and Medicare by patients by having the latest patience test data readily available.

Another advantage of the MB module 103 can involve decreasing patient harm because patient medical records are now easily accessible to patients using an app on their phone or computer. Patients may check for inaccuracies and share information with EHRs.

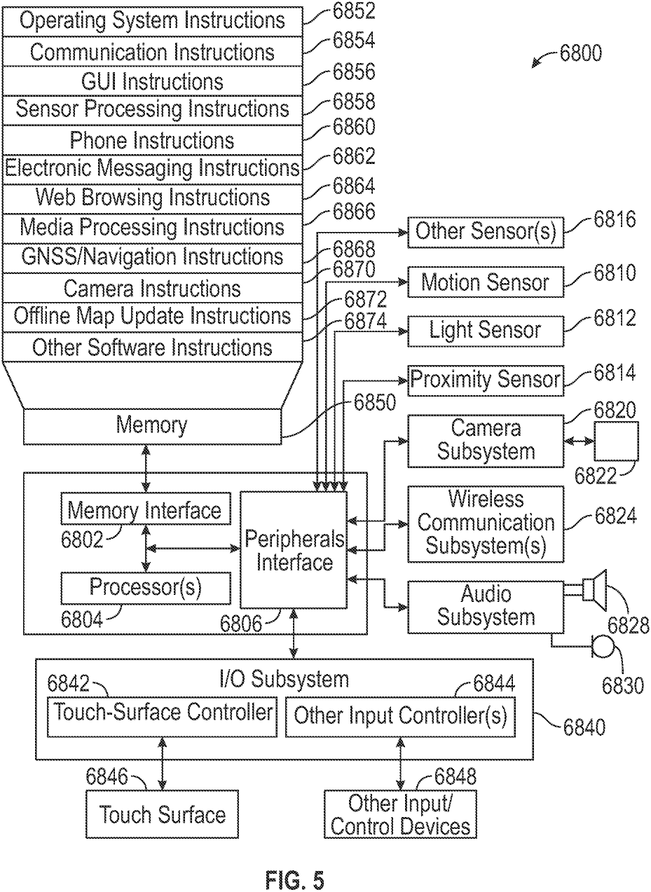

FIG. 5 illustrates a block diagram of an example computing device 6800 that can implement the features, methods, systems, and processes of the embodiments. The computing device 6800 may implement, for example, one or more of the computing devices 309, 321, 319, 311, 313, 315 shown in FIG. 3. The computing device 6800 may be a mobile computing device such as a smartphone or tablet computing device. In some cases, the computing device may be another type of computing apparatus such as a desktop computer or server. The computing device 6800 is presented herein for exemplary and illustrative purposes only and is not considered a limiting feature of the embodiments.

The computing device 6800 depicted in FIG. 5 can include a memory interface 6802, one or more data processors, image processors and/or central processing units 6804, and a peripherals interface 6806. The memory interface 6802, the one or more processors 6804 and/or the peripherals interface 6806 can be separate components or can be integrated in one or more integrated circuits. The various components in the computing device 6800 can be coupled by one or more communication buses or signal lines.

Sensors, devices, and subsystems can be coupled to the peripherals interface 6806 to facilitate multiple functionalities. For example, a motion sensor 6810, a light sensor 6812, and a proximity sensor 6814 can be coupled to the peripherals interface 6806 to facilitate orientation, lighting, and proximity functions. Other sensors 6816 can also be connected to the peripherals interface 6806, such as a global navigation satellite system (GNSS) (e.g., GPS receiver), a temperature sensor, a biometric sensor, magnetometer or other sensing device, to facilitate related functionalities.

A camera subsystem 6820 and an optical sensor 6822, e.g., a charged coupled device (CCD) or a complementary metal-oxide semiconductor (CMOS) optical sensor, can be utilized to facilitate camera functions, such as recording photographs and video clips. The camera subsystem 6820 and the optical sensor 6822 can be used to collect images of a user to be used during authentication of a user, e.g., by performing facial recognition analysis.

Communication functions can be facilitated through one or more wireless communication subsystems 6824, which can include radio frequency receivers and transmitters and/or optical (e.g., infrared) receivers and transmitters. The specific design and implementation of the communication subsystem 6824 can depend on the communication network(s) over which the computing device 6800 is intended to operate. The computing device 6800 may communicate with, for example, the cloud network 109 discussed previously with respect to FIG. 1 and FIG. 2 or the network 307 shown in FIG. 4. Note that in some embodiments network 109 and network 307 may actually be the same network.

For example, the computing device 6800 can include communication subsystems 6824 designed to operate over a GSM network, a GPRS network, an EDGE network, a Wi-Fi or WiMax network, and a Bluetooth™ network. In particular, the wireless communication subsystems 6824 can include hosting protocols such that the device 130 can be configured as a base station for other wireless devices.

An audio subsystem 6826 can be coupled to a speaker 6828 and a microphone 6830 to facilitate voice-enabled functions, such as speaker recognition, voice replication, digital recording, and telephony functions. The audio subsystem 6826 can be configured to facilitate processing voice commands, voiceprinting and voice authentication, for example.

The I/O subsystem 6840 can include a touch-surface controller 6842 and/or other input controller(s) 6844. The touch-surface controller 6842 can be coupled to a touch surface 6846. The touch surface 6846 and touch-surface controller 6842 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch surface 6846.

The other input controller(s) 6844 can be coupled to other input/control devices 6848, such as one or more buttons, rocker switches, thumb-wheel, infrared port, USB port, and/or a pointer device such as a stylus. The one or more buttons (not shown) can include an up/down button for volume control of the speaker 6828 and/or the microphone 6830.

In one implementation, a pressing of the button for a first duration can disengage a lock of the touch surface 6846; and a pressing of the button for a second duration that is longer than the first duration can turn power to the computing device 6800 on or off. Pressing the button for a third duration can activate a voice control, or voice command, module that enables the user to speak commands into the microphone 6830 to cause the device to execute the spoken command. The user can customize a functionality of one or more of the buttons. The touch surface 6846 can, for example, also be used to implement virtual or soft buttons and/or a keyboard.

In some implementations, the computing device 6800 can present recorded audio and/or video files, such as MP3, AAC, and MPEG files. In some implementations, the computing device 6800 can include the functionality of an MP3 player. Other input/output and control devices can also be used. Note that in some situations, the computing device 6800 can be the user device 130 and/or the server device 102.

The memory interface 6802 can be coupled to memory 6850. The memory 6850 can include high-speed random access memory and/or non-volatile memory, such as one or more magnetic disk storage devices, one or more optical storage devices, and/or flash memory (e.g., NAND, NOR). The memory 6850 can store an operating system 6852, such as Darwin, RTXC, LINUX, UNIX, OS X, WINDOWS, or an embedded operating system such as VxWorks.

The operating system 6852 can include instructions for handling basic system services and for performing hardware dependent tasks. In some implementations, the operating system 6852 can be a kernel (e.g., UNIX kernel). In some implementations, the operating system 6852 can include instructions for performing voice authentication. For example, operating system 6852 can implement the NIL application 132 features as described with reference to FIGS. 1-6.

The memory 6850 can also store communication instructions 6854 to facilitate communicating with one or more additional devices, one or more computers and/or one or more servers. The memory 6850 can include graphical user interface instructions 6856 to facilitate graphic user interface processing; sensor processing instructions 6858 to facilitate sensor-related processing and functions; phone instructions 6860 to facilitate phone-related processes and functions; electronic messaging instructions 6862 to facilitate electronic-messaging related processes and functions; web browsing instructions 6864 to facilitate web browsing-related processes and functions; media processing instructions 6866 to facilitate media processing-related processes and functions; GNSS/Navigation instructions 6868 to facilitate GNSS and navigation-related processes and instructions; and/or camera instructions 6870 to facilitate camera-related processes and functions.

The memory 6850 can store other software instructions 6872 to facilitate other processes and functions, such as the venue and indoor map processes and functions as described with reference to FIGS. 1-6 and FIGS. 8-9.

The memory 6850 can also store other software instructions 6874, such as web video instructions to facilitate web video-related processes and functions; and/or web shopping instructions to facilitate web shopping-related processes and functions. In some implementations, the media processing instructions 6866 are divided into audio processing instructions and video processing instructions to facilitate audio processing-related processes and functions and video processing-related processes and functions, respectively.

Each of the above identified instructions and applications can correspond to a set of instructions for performing one or more functions described above. Examples of instructions include the various steps and operations discussed herein with respect to the disclosed methods, systems and devices. These instructions need not be implemented as separate software programs, procedures, or modules. The memory 6850 can include additional instructions or fewer instructions. Furthermore, various functions of the computing device 6800 can be implemented in hardware and/or in software, including in one or more signal processing and/or application specific integrated circuits.

A set of mobile applications (sometimes referred to individually as a “mobile app” or an “app”) can be stored in the memory 6850 (or other persistent storage device (not shown) and can be configured to run on top of device operating system, including by invoking services of the mobile operating system to communicate with networks such as the network 109 and/or the network 308 via a wireless network communication interface with remote resources, such as application servers running applications and/or services with which the mobile app is associated. The mobile operating system and mobile apps have access to and use a memory to store and retrieve data. For example, the mobile operating system may allocate to each app a region of memory to be used by that app to store app-related data. Similarly, each app may be allocated a set of logical locations in a persistent storage managed by the mobile operating system, e.g., an app-specific directory in a file system used by mobile operating system to manage persistently stored objects.

The mobile operating system of the computing device 6800 can be connected to and can manage app interactions with a display subsystem to “display” that can include a touch-sensitive display device, for example, a capacitive or other display able to generate and provide to the mobile operation system, signals representative of single and/or multi-touch gestures, such as swiping (and the direction thereof), pinching in or out, dragging, and dropping. A mobile app may be configured to display app display pages, e.g., app user interface pages, content display pages, etc., via the display associated with the computing device 6800. A mobile app also may be configured to receive user input provided via a display, e.g., selection, dragging, dropping, and/or other user input associated with physical interactions with the touch-sensitive surface of the display.

Note that non-limiting examples of a mobile app and a mobile computing device are disclosed in U.S. Pat. Application Publication No. 20190089826, entitled “Multilayer Mobile App Interface” which issued on Mar. 21, 2019 and is incorporated herein by reference in its entirety.

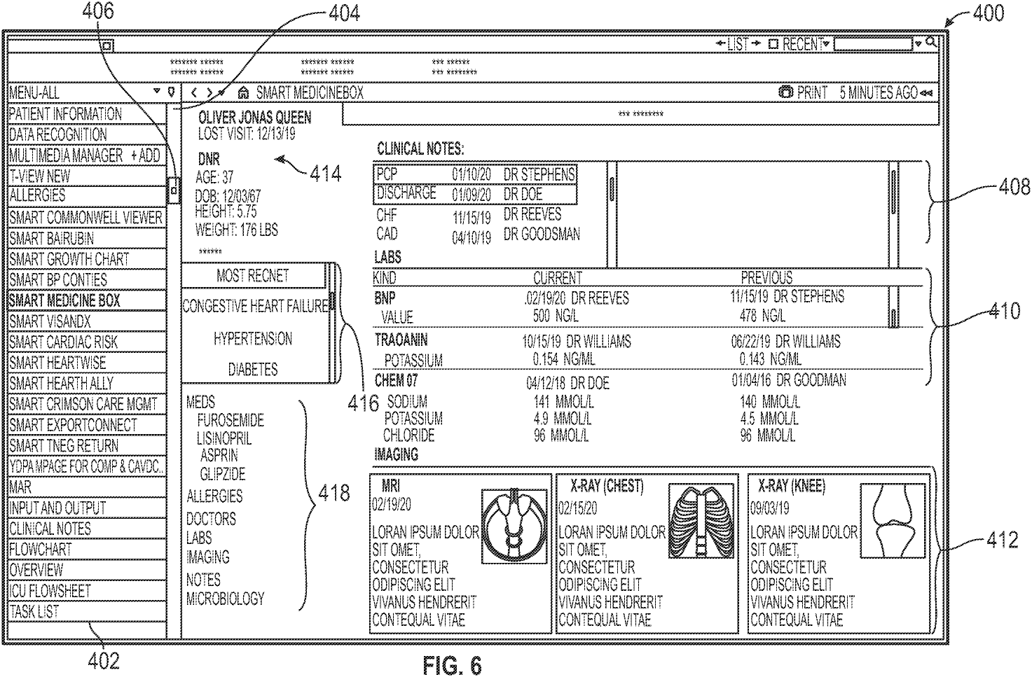

FIG. 6 illustrates a screenshot of a user interface screen 400 in accordance with an embodiment. Note that as utilized herein the term “screenshot” can relate to a screen capture screen grab comprising a digital image that shows the contents of a computer display. A screenshot can be created by the operating system or software running on the device powering the display. The term “user interface” can relate to the point of human-computer interaction and communication in a computing device. A user interface (Ul) can include display screens, keyboards, a pointing device (e.g., a mouse, remote control unit, etc.) and the appearance of a desktop. A Ul is the way through which a user can interact with an application (e.g., an “app”) or a website. A GUI is an example of a Ul.

The user interface screen 400 can be implemented in the context of a GUI ‘window’ or in the context of a mobile app UI (e.g., for smartphones, tablet computing devices, and so on). The user interface screen 400 may include a plurality of sections that can be populated with data related to a patient. In the example shown in FIG. 6, the patient’s name is Oliver Jonas Queen and patient information can be displayed and populated in a GUI section 414. This section can include information about the patient such as the patient’s age, date of birth, etc. A GUI section 416 can include a group of selectable GUI fields labeled Most Recent, Congestive Heart Failure, Hypertension, and Diabetes.

A GUI section 418 displays selectable text such as Meds, Doctors, Labs, Imaging, Notes, and Microbiology. A GUI section 408 is populated with clinical notes, while a GUI section 410 displays the patient’s labs (lab results). Furthermore, a GUI section 412 displays imaging data including, for example, MRI images, and x-ray images. A GUI section 402 can be located at the left hand side of GUI 400 and can contain a menu of plurality of selectable items such as Patient Information, Data Reconciliation and so on. One such selectable item is “SMART MedicineBox”. The GUI section 402 can be embedded in, for example, an EHR GUI. By selecting the item “SMART MedicineBox”, for example, clinically-relevant actionable patient data relevant to the patient Oliver Jonas Queen can be populated in the various GUI sections 408, 410, 412, 414, 406, 416, and 418 in a critical diagnosis hierarchy relevant to the diagnosis of the patient Oliver Jonas Queen.

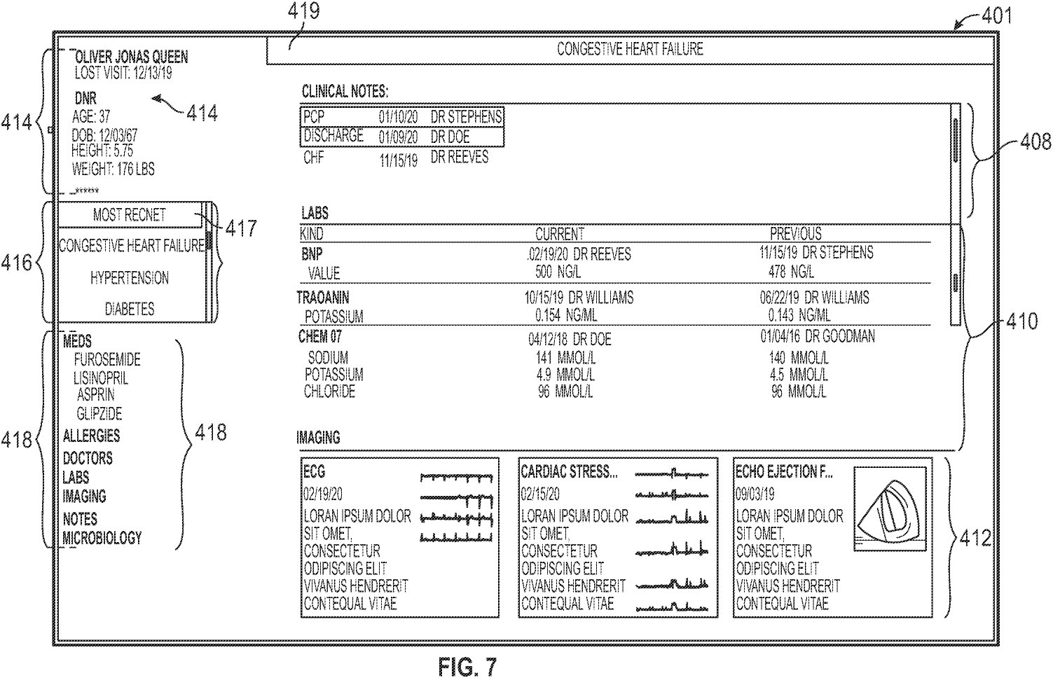

FIG. 7 illustrates a screen shot of a user interface screen 401 in accordance with an embodiment. The user interface screen 401 depicted in FIG. 7 is similar to the user interface screen 402 but without the display of GUI section 402. Imaging data displayed in GUI section 412 include, for example, ECG data, cardiac stress data, and other images or graphs.

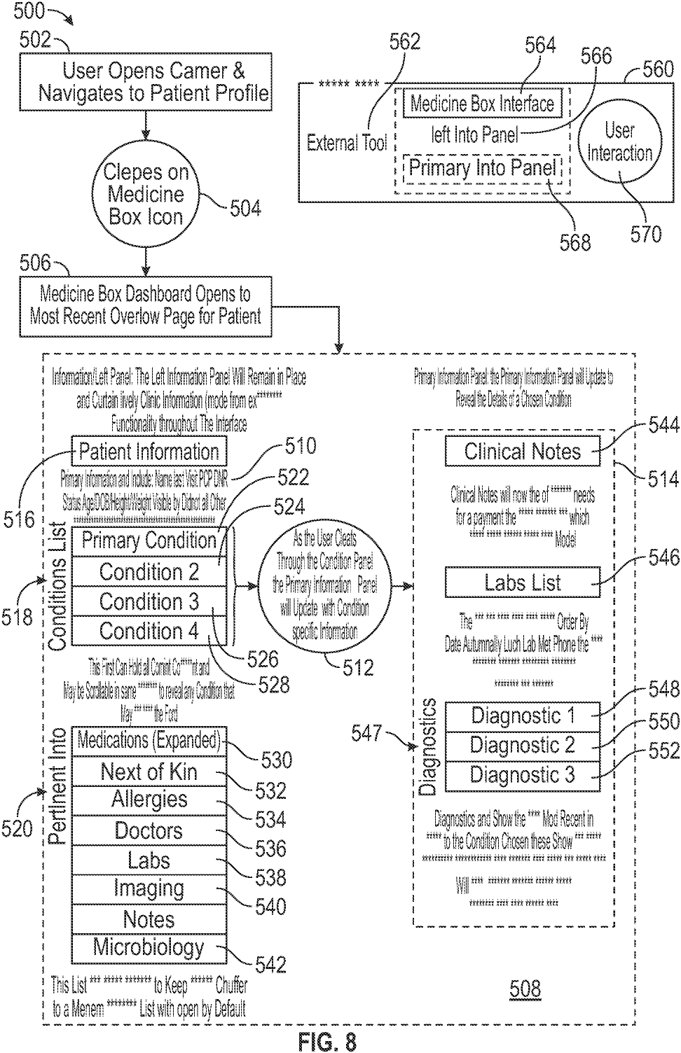

FIG. 8 illustrates a flow diagram 500 depicting user flow for a user interface in which clinically-relevant actionable patient data is automatically populated in response to minimal user input, in accordance with an embodiment. As shown at block 502, a user can open the previously discussed Cerner Code application and navigate to a patient profile. Then, as shown at block 509, the user may ‘click’ (user input) a graphically displayed ‘Medicine Box’ icon. In response to this user input, a Medicine Box graphically displayed dashboard can be opened to the MOST RECENT overview page for the patient, as indicated at block 506. An example of a “MOST RECENT overview page is the GUI screen 400 shown in FIG. 6.

Thereafter, a GUI screen 508 can be displayed (i.e., the aforementioned dashboard) with an information panel 510 that contains various information and sections such as patient information 516, a conditions list 518, and pertinent information 520. The circular block 512 shown in FIG. 7 indicates that as the user clicks through the condition panel shown at the left side of GUI screen 508, the primary information panel 514 can be updated with condition specification information. The information panel 510 can remain in place and can contain fairly static information (aside from expand/collapse functionality) throughout the user interface in which the user interface screen 508 functions. The condition list 518 can hold all current conditions and can be scrollable in some instances to reveal any condition that may fall “below the fold”. In the illustration depicted in FIG. 7, the condition list 518 can list, for example, a primary condition 522, and respective first, second, and third conditions 524, 526, and 528.

The pertinent information 520 may include, for example, medications (expanded) 530, next of kin 532, allergies 534, doctors 536, labs 538, imaging 540, and microbiology 542. The pertinent information 520 list can be expandable/collapsible to keep visual outlier to a minimum. In some embodiments, a medications list associated with the medications (expandable) 530 can open by default.

Note that a GUI legend 560 as depicted in FIG. 8 can represent those items that are an external tool 562 and related to the Medicine Box Ul 564 including items related to the left information panel 566 and the primary information panel 568. A circular block in the legend 560 can relate to the user interaction 570.

A primary information panel 514 can also be displayed in the GUI 508 and contain clinical notes 544, a labs list 546 (“labs”), and diagnostics 547 including diagnostics 548, 550, 552. The primary information panel 514 can automatically update to reveal the details of a selected or chosen medical condition. The labs list 546 can display all recently ordered labs associated with the patient in order by date. Additionally, each displayed lab can show the previous results (if applicable) for comparison. The diagnostics 547 can show the three most recent diagnostic data in relation to the condition chosen including, for examples, thumbnails of any imaging that upon hover can be maximized for ease of viewing.

The disclosed example embodiments are described at least in part herein with reference to flowchart illustrations and/or block diagrams of methods, systems, and computer program products and data structures according to embodiments of the invention. It will be understood that each block of the illustrations, and combinations of blocks, can be implemented by computer program instructions. These computer program instructions may be provided to a processor of, for example, a general-purpose computer, special-purpose computer, or other programmable data processing apparatus to produce a machine, such that the instructions, which execute via the processor of the computer or other programmable data processing apparatus, create means for implementing the functions/acts specified in the block or blocks.

To be clear, the disclosed embodiments can be implemented in the context of, for example a special-purpose computer or a general-purpose computer, or other programmable data processing apparatus or system. For example, in some example embodiments, a data processing apparatus or system can be implemented as a combination of a special-purpose computer and a general-purpose computer. In this regard, a system composed of different hardware and software modules and different types of GUI features may be considered a special-purpose computer designed with the specific purpose of rendering a visualization including the display of data through a GUI. In general, embodiments may be implemented as a method, and/or a computer program product at any possible technical detail level of integration. The computer program product may include a computer readable storage medium (or media) having computer readable program instructions thereon for causing a processor to carry out aspects of the embodiments.

The aforementioned computer program instructions may also be stored in a computer-readable memory that can direct a computer or other programmable data processing apparatus to function in a particular manner, such that the instructions (e.g., steps/operations) stored in the computer-readable memory produce an article of manufacture including instruction means which implement the function/act specified in the various block or blocks, flowcharts, and other architecture illustrated and described herein.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

The flow charts and block diagrams in the figures illustrate the architecture, functionality, and operation of possible implementations of systems, methods, and computer program products according to various embodiments (e.g., preferred or alternative embodiments). In this regard, each block in the flow chart or block diagrams may represent a module, segment, or portion of instructions, which comprises one or more executable instructions for implementing the specified logical function(s).

In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The functionalities described herein may be implemented entirely and non-abstractly as physical hardware, entirely as physical non-abstract software (including firmware, resident software, micro-code, etc.) or combining non-abstract software and hardware implementations that may all generally be referred to herein as a “circuit,” “module,” “engine”, “component,” “block”, “database”, “agent” or “system.” Furthermore, aspects of the present disclosure may take the form of a computer program product embodied in one or more non-ephemeral computer readable media having computer readable and/or executable program code embodied thereon.

It is understood that the specific order or hierarchy of steps, operations, or instructions in the processes or methods disclosed is an illustration of exemplary approaches. For example, the various steps, operations or instructions discussed herein can be performed in a different order. Similarly, the various steps and operations of the disclosed example pseudo-code discussed herein can be varied and processed in a different order. Based upon design preferences, it is understood that the specific order or hierarchy of such steps, operation or instructions in the processes or methods discussed and illustrated herein may be rearranged. The accompanying claims, for example, present elements of the various steps, operations or instructions in a sample order, and are not meant to be limited to the specific order or hierarchy presented.

The inventors have realized a non-abstract technical solution to the technical problem to improve a computer-technology by improving efficiencies in such computer technology. The disclosed embodiments offer technical improvements to a computer-technology such as a data-processing system, and further provide for a non-abstract improvement to a computer technology via a technical solution to the technical problem(s) identified in the background section of this disclosure. The ability to provide patient information, for example, in a fast and efficient manner can lead to improved efficiencies in memory management and processing in the underlying computer technology. The embodiments disclosed herein can provide the benefits of a more seamless GUI implementation along with faster searching of databases and more efficient medical record data storage (i.e., reducing memory usage in a dataprocessing system). Such improvements can result from implementations of the disclosed embodiments. The claimed solution may be rooted in computer technology in order to overcome a problem specifically arising in the realm of computers, computer networks and data processing and data storage management.

Referring again to FIG. 4, it should be noted that the network 307 can communicate bidirectionally with a security module 361, which in turn can communicate bidirectionally with a blockchain system 360, which provides for automatic trust or system trust. The blockchain system 360 is based on the blockchain paradigm, which provides for a decentralized system utilizing decentralized consensus. This can be done in a peer-to-peer manner without an intermediary. The blockchain system 360 can be viewed as a network of nodes running software on a programmable distributed network. It may be referred to as a transaction singleton machine with shared state, a transaction based state machine, a message passing framework, a trustful object messaging compute framework and trusted computing.

A decentralized consensus can be established by a combination of blockchain and cryptography. The security module 361 can provide this cryptography as, for example, a cryptographic unit that requires the use of digital signatures for digital signing applications within, for example, a cryptographic key of an authorized user, with the digital signature being included in a header that can identify each block in the blockchain system 360 as distinct from another block. Each header may include a hash value generated by a hash function. A hash function may be any function that can be used to map input data of arbitrary size to a hash value of a fixed size. Authority and trust can thus be provided by the decentralized virtual network. Consensus logic is generally separate from the application. It may comprise the first layer of a decentralized architecture.

Blockchain utilizes a distributed ledger. A ‘block’ comprises a new group of accepted transactions. A batch of transactions can be released in a block to be validated by the network of participating computers. Continuous, sequential transaction record on a public block creates a unique “chain” or blockchain. This block can be published to all other nodes. The publication can occur periodically, e.g., every 10 minutes.

Note that as utilized herein, the term ‘blockchain’ can relate to any of several types of electronic, computer-based, distributed ledgers. These include consensus-based blockchain and transaction-chain technologies, permissioned and un-permissioned ledgers, shared ledgers and variations thereof. The most widely known application of blockchain technology is the Bitcoin ledger, although other blockchain implementations have been proposed and developed. While Bitcoin may be referred to as a useful application of the technology described herein for the purpose of convenience and illustration, Bitcoin is just one of many applications to which the technology described in the present disclosure may be applied. However, it should be noted that the embodiments are not limited to use with the Bitcoin blockchain and alternative blockchain implementations and protocols, including non-commercial applications, also fall within the scope of the embodiments.

The blockchain system 360 includes the aforementioned blockchain comprising a distributed ledger, which is essentially a database located on disparate nodes. Such a database may be associated with the blockchain system 360. In some embodiments other databases may be utilized for the aforementioned distributed ledger. For example, one or more of the databases 363, 305 and/or other databases may be implemented in different/disparate nodes. Each of the databases 363, 305 and/or other databases may contain the aforementioned distributed ledger and/or portions of the aforementioned distributed ledger. In general, the ledger is a series of sequential blocks. Each node maintains a copy of the distributed ledger, ensuring data integrity, auditability, redundancy, and so on. The blocks making up the distributed ledger each contain records, also known as operations or transactions. The transactions are not distributed by a central authority but are instead constructed by the nodes. In general, transactions are entered in the ledger after being validated or “accepted” by a specified number of nodes. Thus, each node can independently build the ledger from validated transactions, such that all nodes arrive at the same ledger.

The blockchain system 360 (also referred to simply as ‘the blockchain’) is a peer-to-peer, electronic ledger which can be implemented as a computer-based decentralized, distributed system made up of blocks which in turn may be made up of transactions and other information. In some embodiments, a “blockchain transaction” can refer to an input message encoding a structured collection of field values comprising data and a set of conditions, where fulfilment of the set of conditions is prerequisite for the set of fields to be written to a blockchain data structure. For example, with Bitcoin each transaction is a data structure that can encode the transfer of control of a digital asset between participants in the blockchain system 360 and can include at least one input and at least one output. In some embodiments, a “digital asset” can refer to binary data that is associated with a right to use. Examples of digital assets include Bitcoin, ether, Litecoin, and so on. In some implementations, transferring control of a digital asset can be performed by reassociating at least a portion of the digital asset from a first entity to a second entity. Each block of the blockchain system 560 may contain a hash of the previous block so that blocks become chained together to create a permanent, unalterable record of all transactions which have been written to the blockchain since its inception.

The blockchain system 360 can allow the network 307 to function as a decentralized data management platform that is not susceptible to change or variation. That is, the blockchain system 360 can provide a blockchain infrastructure for the network 307. There are generally two categories of blockchains that may be implemented with network 307: permissionless or permissioned. The blockchain system 360 can allow the network 307 to function as either a permissionless blockchain network or a permissioned blockchain network. In an embodiment discussed herein, the network 307 can operate as a permissioned blockchain network, although there may be some situations in which network 307 may operate as a permissionless blockchain network, or which aspects of the network 307 may function as a permissionless blockchain network.

A non-limiting example of a blockchain network, which may be utilized to implement the blockchain system 360 is the blockchain network and distributed ledger disclosed in U.S. Pat. Application Publication No. 20210306135, entitled “Electronic Device Within Blockchain Based PKI Domain, Electronic Device Within Certification Authority Based PKI Domain, and Cryptographic Communication System Including These Electronic Devices,” which published on Sep. 30, 2021 and is incorporated herein by reference in its entirety. In an embodiment, the cryptographic communication system disclosed in U.S. Pat. Application Publication No. 20210306135, including, a public key infrastructure (PKI) based on certification authority (CA) and a blockchain-based PKI, which may be compatible with each other, may be used to implement the security module 361.

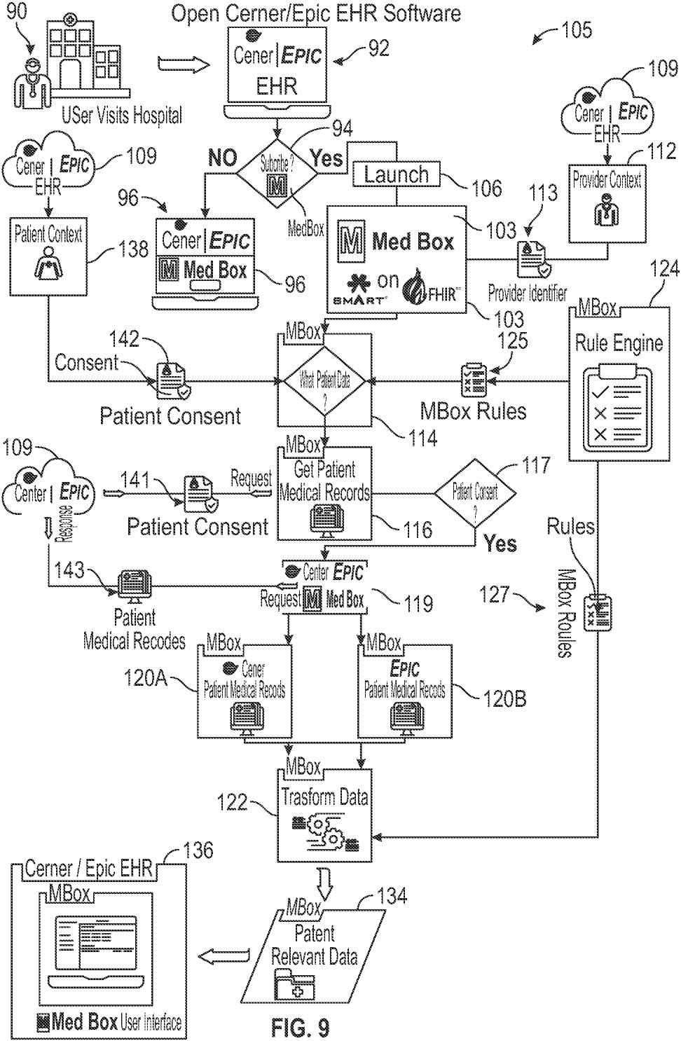

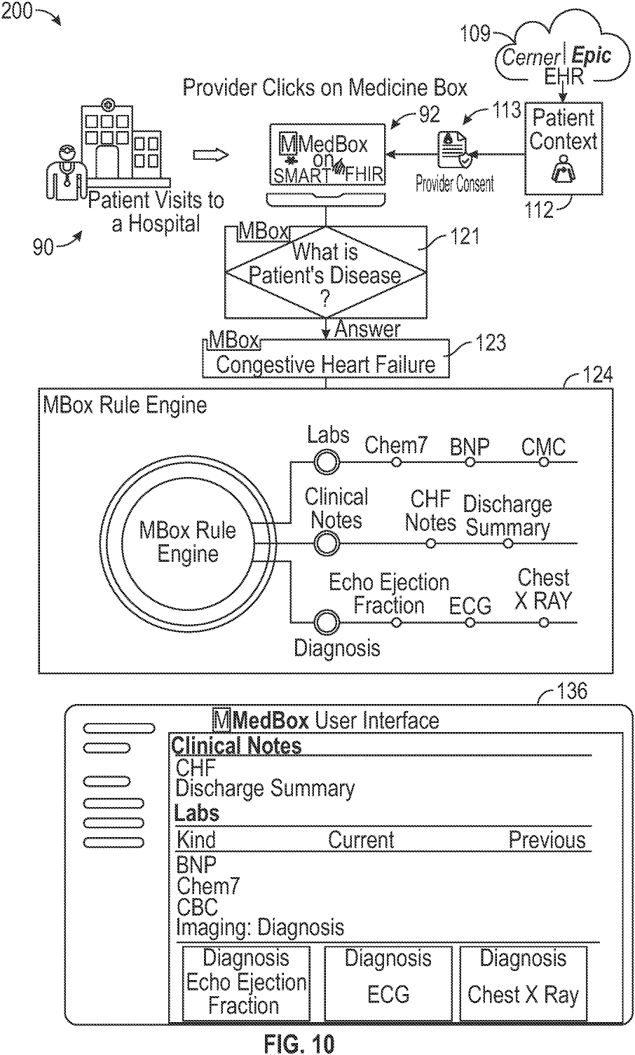

FIG. 9 illustrates a flow chart of operations depicting logical operational steps of a method 105 for providing a user interface for an HER data retrieval system, in accordance with an embodiment. Note that the embodiment of the method 105 depicted in FIG. 9 is similar to the embodiments of the method 100 shown in FIG. 1 and the method 101 depicted in FIG. 2, albeit with some slight differences, which are exemplified in greater detail in the various embodiments discussed below with respect to FIG. 10 to FIG. 17.

FIG. 10 illustrates a flow chart of operations depicting logical operational steps of a method 202 for implementing a rule engine, in accordance with an embodiment. Thus, as discussed previously, and as shown at block 90, a patient may visit a hospital and as shown thereafter, a user (e.g., a physician, nurse practitioner, etc.) may access a medical software platform and/or software application through, for example, a software platform or software application such as open Cerner and/or Epic EHR software through the client device 92 to input and/or view medical data associated with the patient. The client device 92 can be communicatively coupled to the cloud-based network 109, which provides patient context data 112 and patient consent 113.

As shown next at block 121, a query can be input through the client device 92 regarding the patient’s disease or medical condition. In the example shown in FIG. 10, the answer is ‘congestive heart failure’ as shown at block 123. This condition (congestive heart failure) can be input to the rule engine 124, which in the example shown in FIG. 10, can generate medical data based on specific rules with respect to the identified condition (congestive heart failure). In the example, shown in FIG. 10, the rule engine 124 can generate labs including Chem7, BNP and CBC related to the congestive heart feature condition, for display in a user interface 136.

The rule engine 124 may also generate and display clinical notes tied to the congestive heart failure condition including, for example, CHF notes and a discharge summary. The rule engine 124 may also generate diagnosis data, such as an echo ejection fraction, ECG, and a chest x-ray, which relate to the identified condition (congestive heart failure). It should be appreciated that reference to the congestive heart failure condition as shown in FIG. 10 is for exemplary purposes only and that other types of patient medical conditions would trigger different rules for processing by the rule engine, resulting in the generation and display of different medical data related to these other types of medical conditions. The user interface 136 shown in FIG. 10 thus displays medical data related to the congestive heart failure condition.

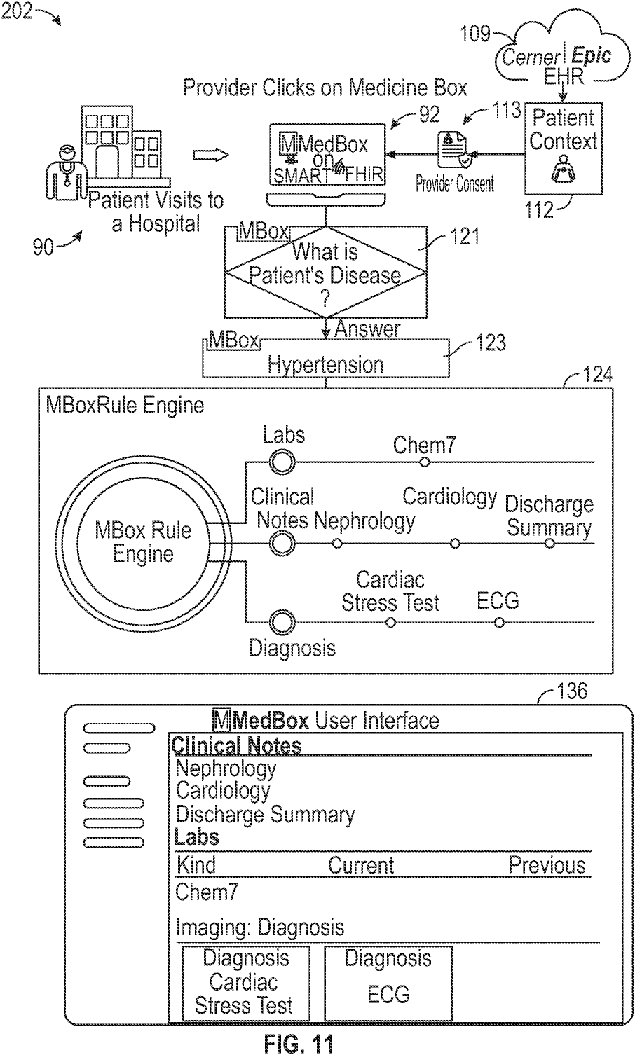

FIG. 11 illustrates a flow chart of operations depicting logical operational steps of a method 202 for implementing the rule engine 124, in accordance with an embodiment. In the example depicted in FIG. 11, the question asked with respect to decision block 121 results in an answer of a medical condition of hypertension. That is, as shown at block 121, a determination is made as to the patient’s disease. In this case, hypertension is identified as shown at block 123, and then supplied to the rule engine 124 which generates data for display based on the specific condition of hypertension.

Note that similar or identical reference numerals as utilized herein refer generally to identical elements or features, albeit with different functionalities and components. For example, the rule engine 124 shown in FIG. 11 operates according to rules that are different from the rule engine 124 depicted in FIG. 10. That is, the rule engine 124 depicted in FIG. 10 shows includes slightly different rules such as displaying clinical notes including CHF notes and a discharge summary, while the clinical notes shown in FIG. 11 with respect to the rule engine 124 includes notes related to nephrology, cardiology, and the discharge summary. The diagnosis of the rule engine 124 depicted in FIG. 11 includes information such as a cardiac stress test and ECG data, while the diagnosis data of the rule engine 124 depicted in FIG. 10 includes diagnosis data such as an echo ejection fraction, ECG data and a chest X-ray. The labs of the rule engine 124 shown in FIG. 11 include Chem7 data, whereas the labs of the rule box engine 124 of FIG. 10 include Chem7 data, BNP and CBC data. In other word FIG. 11 depicts a different embodiment than that shown in FIG. 10.

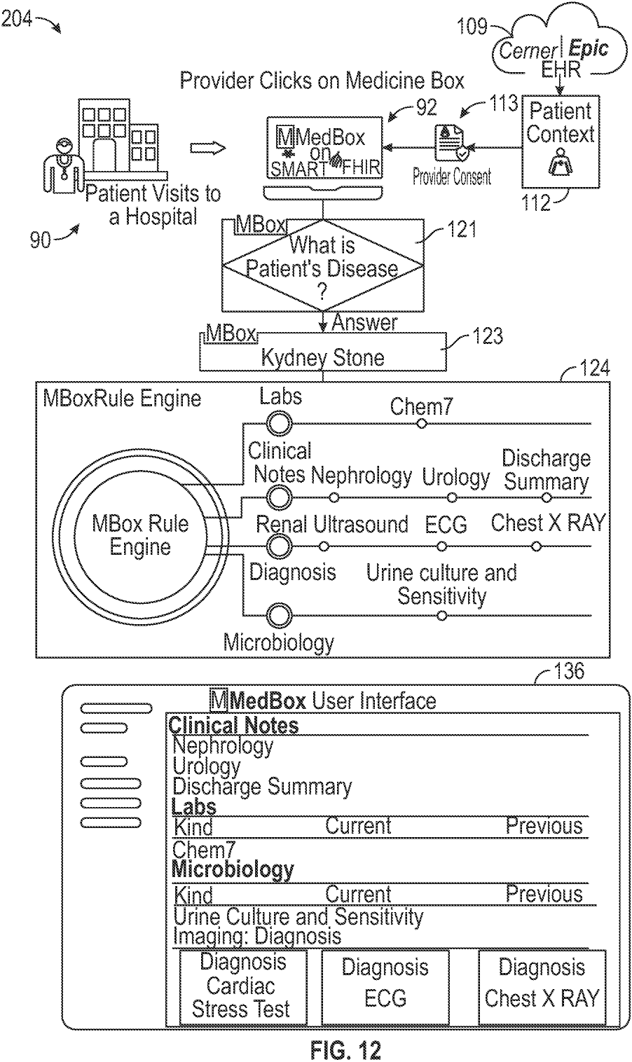

FIG. 12 illustrates a flow chart of operations depicting logical operational steps of a method 204 for implementing the rule engine 124, in accordance with an embodiment. The method 204 illustrated in FIG. 12 is similar to the method 200 depicted in FIG. 10 and the method 202 shown in FIG. 11, albeit with a medical condition of ‘kidney stone’ as shown at block 123. The rule engine 124 and the user interface 136 can thus generate and display medical data related to the patient’s kidney stone condition. In the example rule engine 124 depicted in FIG. 12, rules for generating and displaying data include lab data such as Chem7, clinical notes including nephrology, urology, and a discharge summary. Diagnosis data can include, for example, renal ultrasound data, ECG, and a chest X-ray. Microbiology data can include, for example, urine culture and sensitivity data. This information can be accessed and viewed by a user through the user interface 136.

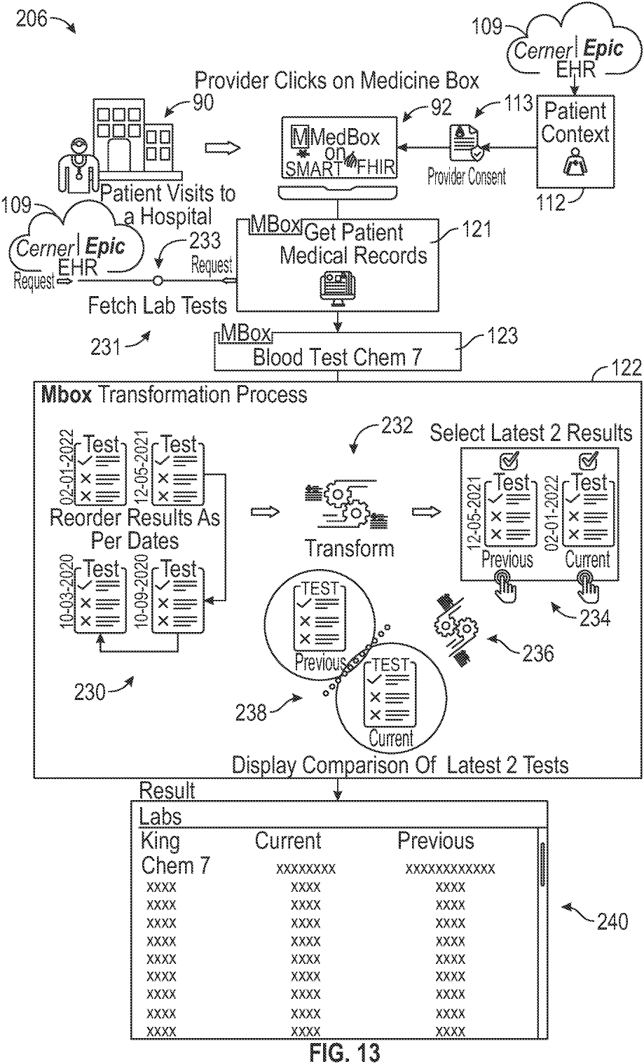

FIG. 13 illustrates a flow chart of operations depicting logical operational steps of a method 206 for implementing a transformation process 122, in accordance with an embodiment. Note that some of the operations shown in FIG. 13 have already been described herein. In the interest of brevity these operations will not be described again. Nevertheless, in response to a request to obtain medical records as shown at block 121, lab tests can be fetched from the cloud-based network 109. In the example shown in FIG. 13, a blood test Chem7 is fetched, as shown at block 123 and subject to the transformation process 122.

In the example embodiment depicted in FIG. 13, the transformation process 122 can involve a reordering 230 of the lab test results as per dates, which can be then subject to a data transformation 232 (i.e., transformation process), which can result in the selection of the latest two results, as shown at block 234, followed by another transformation process 236, which can result in a display 238 of a comparison of the latest two tests. These results can then be displayed in a user interface window 240 of the user interface 136.

Note that the term ‘window’ as utilized herein can relate to a GUI window and GUI features such as a GUI section that graphically displays selectable data in a textual listing format and/or a graphical object format. Such selectable data can be selected used to drill down further for a listing and display of additional data (e.g., data of increasing granularity) and/or other features such as GUI icons, cursors, buttons, selectable text, objects, and so on. The term ‘window’ may also relate in some instances to a menu or list of selectable menu items. The term ‘window’ as utilized herein can also refer to the element (or elements) that can information/data on a GU I screen.

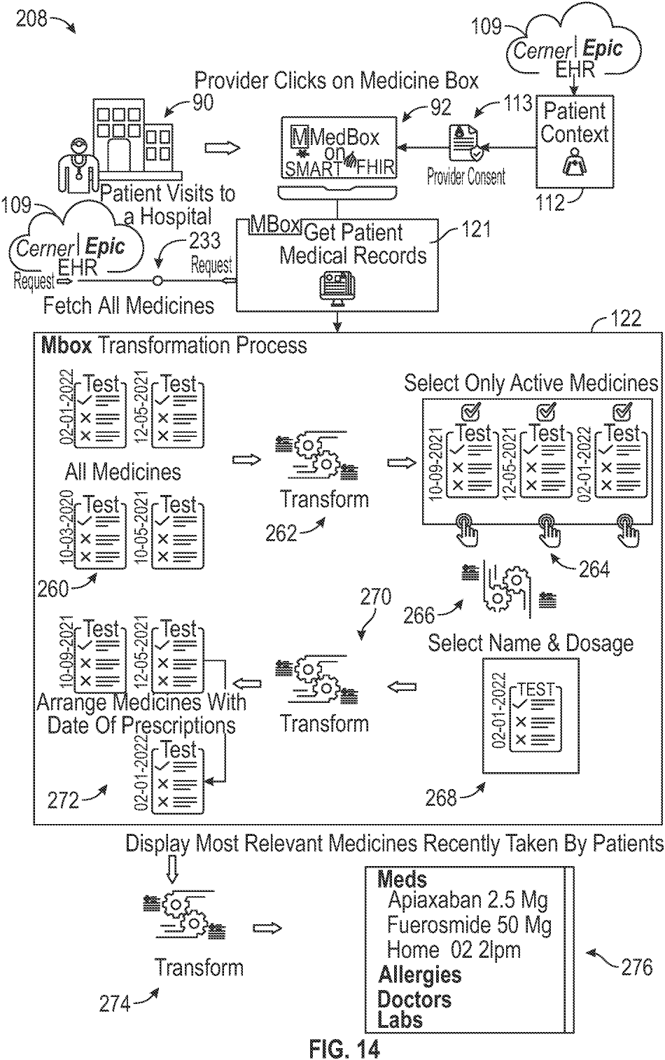

FIG. 14 illustrates a flow chart of operations depicting logical operational steps of a method 208 for implementing a transformation process, in accordance with an embodiment. Note that the transformation process example shown in FIG. 14 can involve a request for patient medical records as shown at block 121, which in this case is a request to fetch data indicative of all medicines associated with the patient. This data (e.g., prescription data) can be them subject to the transformation process 122 shown in FIG. 14, which can involve identifying all medicines/prescriptions 260, which are subject to a transformation process 262, which then can result in a selection of only active medicines as shown at block 264. The results from the operation depicted at block 264 are then subject to a transformation process 266, which allows for a selection of a medicine name and dosage and as shown at block 268. Following process of the operation indicated at block 268, another transformation process 270 can be implemented with respect to the selected medicines, following by implementation of an arrangement operation 282 in which the medicines are automatically arranged by the date of prescription(s). The selected/active medicines are then subject to another transformation process/operation 274, and the resulting data can be displayed in window 276 of the user interface 136 as the most relevant medicines recently taken by the patient. For example, in window 276, selectable fields can be displayed including “Meds”, “Allergies”, “Doctors”, and “Labs”.

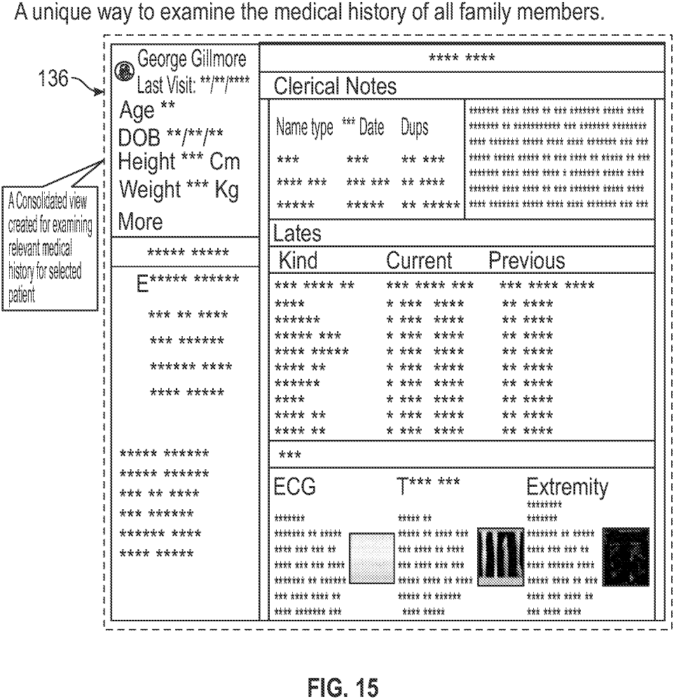

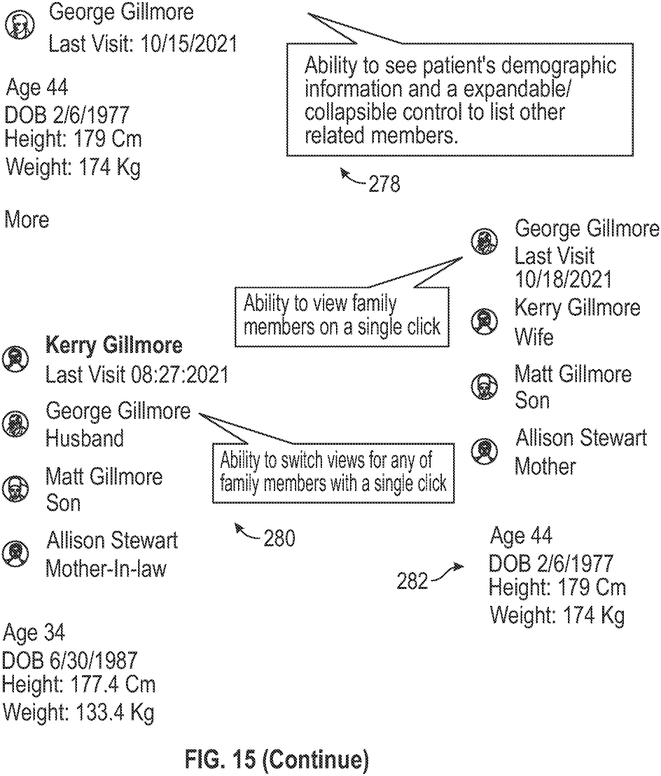

FIG. 15 illustrates a diagram depicting a user interface 136 and associated user interface features, in accordance with an embodiment. The user interface 136 as shown in FIG. 15 can provide a consolidated view for examining the relevant history for a selected patient, and also for examining the medical history of all family members. Window 278 can allow for a user with the ability to view a patient’s demographic information and an expandable/collapsible control to list other related members, which as shown in window 280 and window 282. Window 280 can, for example, provide a user with the ability to switch views for any family member with a single click. Window 282 allows a user to view family members on a single click.

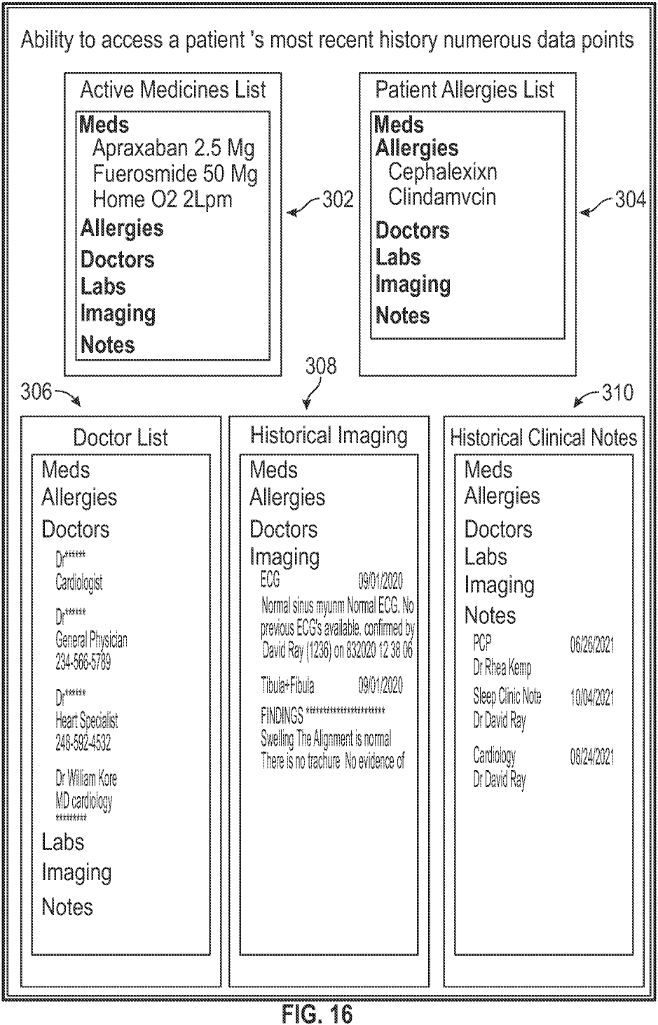

FIG. 16 illustrates various types of data that can be displayed by the user interface 136, in accordance with an embodiment. In the example embodiment shown in FIG. 16, a window 302 can display an active medicines list, and a window 304 can display a patient’s allergies list. Window 306 can display historical imaging data and window 310 can display historical clinical notes.

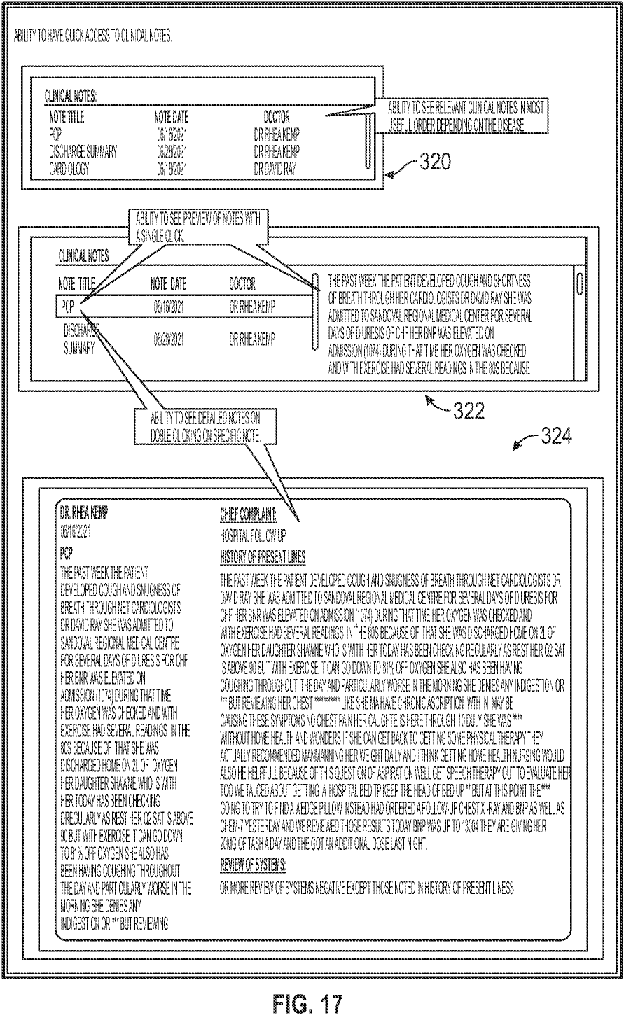

FIG. 17 illustrates additional types of data that can be displayed by the user interface 136, in accordance with an embodiment. The data shown in FIG. 17 relates to the ability of users to have quick access to clinical notes. For example, window 320 can provide a user with the ability to see relevant clinical notes in the most useful order depending on the disease. Window 322 can provide a user with the ability to see a preview of notes with a single click, and the ability to see notes upon double clicking a specific note. The ability to see notes upon double clicking a specific note is also a feature of window 324.

Certain methods or process blocks may be omitted in some implementations. The methods and processes described herein are also not limited to any particular sequence, and the blocks or states relating thereto can be performed in other sequences that may be appropriate. For example, described blocks or states may be performed in an order other than that specifically disclosed, or multiple blocks or states may be combined in a single block or state. The example blocks or states may be performed in serial, in parallel or in some other manner. Blocks or states may be added to or removed from the disclosed example embodiments.

It will also be appreciated that various items are illustrated as being stored in memory or on storage while being used, and that these items or portions thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc.

Some or all of the modules, systems and data structures may also be stored (e.g., as software instructions or structured data) on a computer-readable medium, such as a hard disk, a memory, a network or a portable media article to be read by an appropriate drive or via an appropriate connection. The systems, modules and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some or all of the elements in the list.

While certain example embodiments have been described, these embodiments have been presented by way of example only and are not intended to limit the scope of the inventions disclosed herein. Thus, nothing in the foregoing description is intended to imply that any particular feature, characteristic, step, module or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method for self-populating a user interface with medical data, comprising:
transforming patient data associated with a patient into clinically-relevant actionable patient data that is relevant to the patient based on at least one diagnosis of the patient and which is displayable in a screen of a user interface of an electronics health records (EHR) system in a critical diagnosis hierarchy relevant to the at least one diagnosis of the patient; and
in response to a user input, automatically populating the screen of the user interface with the clinically-relevant actionable patient data according to the critical diagnosis hierarchy and the at least one diagnosis in a manner that minimizes the user input with respect to the user interface.

2. The method of claim 1 further comprising:
fetching the patient data associated with the patient and based on the at least one diagnosis of the patient.

3. The method of claim 1 further comprising:
fetching the patient data associated with the patient and based on the at least one diagnosis of the patient prior to transforming the patient data into the clinically-relevant actionable patient data.

4. The method of claim 1 wherein the at least one diagnosis is derived from a medical ontology.

5. The method of claim 1 wherein the user interface comprises a touch-sensitive display.

6. The method of claim 1 wherein the clinically-relevant actionable patient data displayed in the user interface includes at least one of:
imaging data relevant to the patient;
medical images relevant to the patient;
test results relevant to the patient;
laboratory results relevant to the patient;
medical allergies relevant to the patient; and
prescriptions relevant to the patient.

7. A method for self-populating a user interface with medical data, comprising:
deriving at least one diagnosis of a patient from a medical ontology;
transforming patient data associated with the patient into clinically-relevant actionable patient data that is relevant to the patient based on the at least one diagnosis of the patient and which is displayable in a screen of a user interface of an electronics health records (EHR) system in a critical diagnosis hierarchy relevant to the at least one diagnosis of the patient; and
automatically populating the screen of the user interface with the clinically-relevant actionable patient data according to the critical diagnosis hierarchy and the at least one diagnosis in a manner that minimizes user input with respect to the user interface.

8. The method of claim 7 further comprising:
fetching the patient data associated with the patient and based on the at least one diagnosis of the patient prior to transforming the patient data into the clinically-relevant actionable patient data.

9. The method of claim 7 wherein the clinically-relevant actionable patient data displayed in the user interface includes at least one of:
imaging data relevant to the patient;
medical images relevant to the patient;
test results relevant to the patient;
laboratory results relevant to the patient;
medical allergies relevant to the patient; and
prescriptions relevant to the patient.

10. The method of claim 9 further comprising:
fetching the patient data associated with the patient and based on the at least one diagnosis of the patient prior to transforming the patient data into the clinically-relevant actionable patient data.

11. A system for self-populating a user interface with medical data, the system comprising:
at least one processor and a memory storing processor-executable codes, wherein the at least one processor is configured to implement the following operations upon executing the processor-executable codes:
transforming patient data associated with a patient into clinically-relevant actionable patient data that is relevant to the patient based on at least one diagnosis of the patient and which is displayable in a screen of a user interface of an electronics health records (EHR) system in a critical diagnosis hierarchy relevant to the at least one diagnosis of the patient; and
in response to a user input, automatically populating the screen of the user interface with the clinically-relevant actionable patient data according to the critical diagnosis hierarchy and the at least one diagnosis in a manner that minimizes the user input with respect to the user interface.

12. The system of claim 11 wherein the processor-executable codes are configured to fetch the patient data associated with the patient and based on the at least one diagnosis of the patient.

13. The system of claim 11 wherein the processor-executable codes are configured to fetch the patient data associated with the patient and based on the at least one diagnosis of the patient prior to transforming the patient data into the clinically-relevant actionable patient data.

14. The system of claim 11 wherein the at least one diagnosis is derived from a medical ontology.

15. The system of claim 11 wherein the user interface comprises a touch-sensitive display.

16. The system of claim 1 wherein the clinically-relevant actionable patient data displayed in the user interface includes at least one of:
imaging data relevant to the patient;
medical images relevant to the patient;
test results relevant to the patient;
laboratory results relevant to the patient;
medical allergies relevant to the patient; and
prescriptions relevant to the patient.

17. (canceled)

18. (canceled)

19. (canceled)

20. (canceled)
